(19) United States
(12) Patent Application Publication
(10) Pub. No.: US 2026/0100267 A1
(43) Pub. Date: Apr. 9, 2026

(54) Systems and Methods for Automated Diagnosis of Disease Related Risk Factors in 3D Biomedical Imaging

(71) Applicant: The Regents of the University of California, Oakland, CA (US)

(72) Inventors: Oren Avram, Los Angeles, CA (US); Berkin Durmus, Oakland, CA (US); Nadav Rakocz, Oakland, CA (US); Jeffrey Chiang, Los Angeles, CA (US); Srinivas Sadda, Oakland, CA (US); Eran Halperin, Santa Monica, CA (US)

(73) Assignee: The Regents of the University of California, Oakland, CA (US)

(21) Appl. No.: 19/156,782

(22) PCT Filed: Feb. 14, 2024
(86) PCT No.: PCT/US2024/015861

§ 371 Date: Aug. 14, 2025

Related U.S. Application Data
(60) Provisional application No. 63/484,946, filed on Feb. 14, 2023.

(51) Int. Cl. G16H 30/40; G06N 3/045; G06N 20/00; G06V 10/774; G16H 50/20; G16H 50/30; G16H 50/70

(52) U.S. Cl. CPC G16H 30/40; G06N 3/045; G06N 20/00; G06V 10/774; G16H 50/20; G16H 50/30; G16H 50/70

(57) ABSTRACT

Deep learning methods and systems for detecting biomarkers within volumetric biomedical imaging dataset using such deep learning methods and systems are provided. Embodiments predict the clinically useful biomarkers in optical coherent tomography images, ultrasound images, magnetic resonance imaging images, and computed tomography images using deep neural networks.

### CROSS-REFERENCE TO RELATED APPLICATIONS

The current application claims the priority to U.S. Provisional Patent Application No. 63/484,946 entitled “Systems and Methods for Automated Diagnosis of Disease Related Risk Factors in 3D Biomedical Imaging” filed Feb. 14, 2023. The disclosure of U.S. Provisional Patent Application No. 63/484,946 is hereby incorporated by reference in its entirety for all purposes.

### FIELD OF THE INVENTION

The current disclosure is directed to deep learning methods and systems capable of detecting and classifying disease related risk factors; and more particularly to methods and systems for diagnosing disease related risk factors in 3D biomedical images using such deep learning methods and systems.

### BACKGROUND

Biomedical imaging analysis is an important component of clinical care with applications across multiple domains. For example, analyzing optical coherence tomography (OCT) images of the retina allows ophthalmologists to diagnose and follow up on ocular diseases, such as age-related macular degeneration (AMD), and tailor appropriate and personalized interventions to delay the progression of retinal atrophy and irreversible vision loss. Another example is the analysis of heart function using cardiac imaging, such as heart computed tomography (CT) and ultrasound. Monitoring heart function can help cardiologists assess potential cardiac issues, prescribe medications to improve a medical condition, e.g., reduced heart ejection fraction, and guide treatment decisions. Radiologists' analysis and regular monitoring of breast imaging such as mammography and magnetic resonance imaging (MRI) help detect early breast cancers, initiate a consequent interventive therapy, and determine the effectiveness of such therapeutics. These medical insights and actionable information are obtained following an expert's time-intensive manual analysis. The automation of these analyses using artificial intelligence may improve healthcare as it reduces costs and treatment burden.

### SUMMARY OF THE INVENTION

Systems and methods in accordance with some embodiments of the invention are directed to deep learning methods and systems for detecting and classifying disease related risk factors.

Some embodiments include a method to predict biomarkers in biomedical imaging, comprising: reshaping a plurality of three-dimensional images into a plurality of two-dimensional images by stacking a plurality of slices of said three-dimensional images on top of one another using a computer system; applying a pre-trained feature extractor to the plurality of two-dimensional images, wherein the pre-trained feature extractor independently operates on each of the plurality of two-dimensional images, and generates a plurality of feature maps; applying a convolutional neural network to operate across the plurality of feature maps, wherein the convolutional neural network produces a feature vector; and generating an output of biomarker prediction, wherein the prediction is a transformation of the feature vector; wherein the plurality of three-dimensional images is selected from a group consisting of: optical coherent tomography images, ultrasound images, magnetic resonance imaging images, and computed tomography images.

In some embodiments, the biomarker for optical coherent tomography images is selected from the group consisting of drusen volume (DV), intraretinal hyperreflective foci (IHRF), subretinal drusen deposits (SDD), hyporeflective drusen core (hDC), and any combinations thereof.

In some embodiments, the biomarker for ultrasound images comprises ejection fraction, cardiomyopathy, and a combination thereof.

In some embodiments, the biomarker for magnetic resonance imaging images comprises hepatic proton density fat fraction level.

In some embodiments, the biomarker for computed tomography images comprises nodule malignancy in thoracic cancer.

Some embodiments further comprise: obtaining a training dataset of images using the computer system; generating a first set of features for each image in the training dataset based upon object classification using the computer system; and training the feature extractor to learn relationships between the set of images in the training dataset and the first set of features in the training dataset using the computer system.

In some embodiments, the training dataset comprises a plurality of ImageNet dataset.

Some embodiments further comprise: obtaining an annotated training dataset comprising optical coherent tomography images using the computer system, wherein each of the optical coherent tomography images is annotated with at least one retinal disease risk factor; generating a second set of features for each annotated image in the annotated training dataset using the computer system; and training the feature extractor to learn relationships between each annotated image and the second set of features in the annotated training dataset using the computer system.

In some embodiments, the annotated training dataset comprises two-dimensional images of optical coherent tomography images.

In some embodiments, the two-dimensional images are fovea scans.

In some embodiments, the pre-trained feature extractor trained with the annotated training dataset of optical coherent tomography images is used for analyzing three-dimensional images selected from a group consisting of: optical coherent tomography images, ultrasound images, magnetic resonance imaging images, and computed tomography images, and generating biomarker predictions thereof.

In some embodiments, the plurality of slices is stacked linearly to form the two-dimensional image.

In some embodiments, the convolutional neural network comprises a vision transformer module.

In some embodiments, the feature vector transformation is a decision layer comprising at least two fully connected layers.

Some embodiments include a method of training a feature extractor, comprising: obtaining a training dataset of images using a computer system; generating a first set of features for each image in the training dataset based upon object classification using the computer system; and training a feature extractor model to learn relationships between the set of images in the training dataset and the first set of features in the training dataset using the computer system.

In some embodiments, the training dataset comprises a plurality of ImageNet dataset.

Some embodiments further comprise: obtaining an annotated training dataset of optical coherent tomography images using the computer system, wherein each of the optical coherent tomography images is annotated with at least one retinal disease risk factor; generating a second set of features for each annotated image in the annotated training dataset using the computer system; and training the feature extractor model to learn relationships between each annotated image and the second set of features in the annotated training dataset using the computer system.

In some embodiments, the annotated training dataset comprises two-dimensional images of optical coherent tomography images.

In some embodiments, the two-dimensional images are fovea scans.

In some embodiments, the feature extractor trained with the annotated training dataset of optical coherent tomography images is used for analyzing three-dimensional images selected from a group consisting of: optical coherent tomography images, ultrasound images, magnetic resonance imaging images, and computed tomography images, and generating biomarker predictions thereof.

In some embodiments, the biomarker for optical coherent tomography images is selected from the group consisting of drusen volume (DV), intraretinal hyperreflective foci (IHRF), subretinal drusen deposits (SDD), hyporeflective drusen core (hDC), and any combinations thereof; wherein the biomarker for ultrasound images comprises ejection fraction, cardiomyopathy, and a combination thereof; wherein the biomarker for magnetic resonance imaging images comprises hepatic proton density fat fraction level; wherein the biomarker for computed tomography images comprises nodule malignancy in thoracic cancer.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the following figures, which are presented as embodiments of the invention and should not be construed as a complete recitation of the scope of the invention, wherein:

FIG. 1 illustrates a schematic of SLViT process in accordance with an embodiment.

FIG. 2 illustrates performance overview of the SLIViT processes in accordance with an embodiment.

FIGS. 3A and 3B illustrate ROC AUC performance comparison of five models in accordance with an embodiment.

FIGS. 4A and 4B illustrate PR AUC performance comparison of five models in accordance with an embodiment.

FIGS. 5A through 5C illustrate performance comparison on cardiac function prediction tasks using echocardiograms in accordance with an embodiment.

FIG. 6 illustrates performance comparison of a cardiomyopathy binary classification task on echocardiograms in accordance with an embodiment.

FIGS. 7A through 7D illustrate SLIViT's ROC curve compared to junior clinical retina specialists' assessment in accordance with an embodiment.

FIGS. 8A through 8D illustrate SLIViT's PR performance compared to junior clinical retina specialists' assessment in accordance with an embodiment.

FIG. 9 illustrates SLIViT's performance in a volumetric-OCT frame-permutation experiment in accordance with an embodiment.

FIGS. 10A and 10B illustrate pre-training ablation study for (volumetric) OCT-related downstream learning tasks in accordance with an embodiment.

FIG. 11 illustrates pre-training ablation study for (volumetric) non-OCT-related downstream learning tasks in accordance with an embodiment.

FIGS. 12A through 12I illustrate feature similarity analysis between various pre-trained backbones in accordance with an embodiment.

FIG. 13 illustrates 2D biomedical-imaging pre-training performance contribution for 3D OCT-related downstream learning tasks in accordance with an embodiment.

FIG. 14 illustrates 2D biomedical-imaging pre-training performance contribution for 3D non-OCT-related downstream learning tasks in accordance with an embodiment.

FIG. 15 illustrates an architecture ablation study in accordance with an embodiment.

FIGS. 16A through 16D illustrate ViT hyperparameter tuning experiment in accordance with an embodiment.

FIG. 17 illustrates a computer system that can be configured to implement any computing system in accordance with an embodiment.

### DETAILED DESCRIPTION OF THE INVENTION

Training artificial intelligence models to accurately detect clinical features in biomedical imaging may require a large dataset of annotated biomedical imaging. However, the existence of annotated volumetric biomedical imaging datasets, such as 3D magnetic resonance imaging (MRI) scans, 3D optical coherence tomography (OCT) scans, and ultrasound videos, is limited. Thus 3D-based vision models may be bounded by a performance limit due to the lack of annotated 3D data.

Deep vision models, such as Convolutional Neural Networks (CNNs) and their derivatives can be used to tackle computer vision tasks and medical-related vision tasks. In order to train a deep vision model to accurately learn and predict a target variable in a general vision task (excluding segmentation tasks) from scratch, a large number of training samples is needed. Transfer learning may address this challenge by pre-training a vision model for a general learning task on a large data set, and then using this general model as a starting point for training a specialized model on a smaller dataset. An advantage of transfer learning is that the pre-training can be done on a large dataset in a domain where data are abundant, and then the fine-tuning can be done using a small dataset in the domain of interest. Using a transfer learning approach, deep vision methods analyzing 2D biomedical-imaging were first pre-trained on over a million labeled natural images (in a supervised fashion), and then fine-tuned to a specific medical-learning task on a smaller number of labeled biomedical images (for example fewer than 10,000). (See, e.g., McKinney, S. M. et al., Nature 577, 89-94 (2020); Hannun, A. Y. et al., Nat. Med. 25, 65-69 (2019); Rajpurkar, P. et al., PLoS Med. 15, e1002686 (2018); Gulshan, V. et al., JAMA 316, 2402-2410 (2016); Deng, J. et al., IEEE Conference on Computer Vision and Pattern Recognition 248-255 (2009); the disclosures of which are incorporated by reference.) Some methods used self-supervised-based transfer-learning techniques relying mainly on unlabeled medical data, and others combined both natural and medical images. (See, e.g., Tiu, E. et al., Nat Biomed Eng 6, 1399-1406 (2022); Zhang, Y., et al., Contrastive Learning of Medical Visual Representations from Paired Images and Text. arXiv (2020); Xie, Y., et al., UniMiSS: Universal Medical Self-Supervised Learning via Breaking Dimensionality Barrier. arXiv (2021); Azizi, S. et al. Big Self-Supervised Models Advance Medical Image Classification. arXiv (2021); the disclosures of which are incorporated by reference.) The pre-trained weights can be leveraged as ‘prior knowledge’ for fine-tuning downstream learning tasks in these 2D biomedical imaging deep vision models.

Medical diagnoses may rely on volumetric biomedical imaging (e.g., 3D OCT scans, MRI scans, or ultrasound videos). Transfer learning may not be directly applicable to 3D biomedical imaging, since in contrast to the 2D domain, there is no large annotated dataset of structured 3D scans. Moreover, annotating 3D biomedical images is far more labor-prohibitive than 2D images. For example, a 3D OCT scan that includes 97 2D frames (usually referred to as B-scans) can require a 5-10 minutes inspection of a highly trained clinical retina specialist in order to detect retinal-disease biomarkers such as the volume of a drusen lesion. Therefore, considering the resources devoted to annotating 3D biomedical images, it is practically infeasible to annotate 100,000 (or more) volumes, to eliminate the necessity of supervised transfer learning. In fact, merely compiling such large-sized volumetric datasets (without labels) that are needed for self-supervised-based learning could be cost-, processing-, and storage-prohibitive when standard resources are available. These gaps are acute because supervised models for 3D image analysis, such as 3D ResNet and 3D Vision Transformer (ViT), involve the optimization of a large number of parameters, thus requiring large datasets for training.

Several attempts have been undertaken to tackle volumetric-biomedical-imaging learning tasks with sparsely annotated training datasets on different data modalities. For instance, SLIVER-net was designed for binary classification of AMD biomarkers in 3D OCT scans. EchoNet was designed to predict heart ejection fraction (EF) in echocardiograms. 2D-Slice-CNN-based methods and 3D ResNet-based architectures were used in 3D MRI scans in diagnosing Alzheimer's disease, breast cancer, and Parkinson's disease. (See, e.g., Rakocz, N. et al., NPJ Digit Med 4, 44 (2021); Ghorbani, A. et al. NPJ Digit Med 3, 10 (2020); Gupta, U. et al. Transferring Models Trained on Natural Images to 3D MRI via Position Encoded Slice Models. ArXiv (2023); Witowski, J. et al., Sci. Transl. Med. 14, eabo4802 (2022); Yang, M., et al., Biomed. Signal Process. Control 85, 104904 (2023); the disclosures of which are incorporated by reference.) 3D ResNet is considered a solid baseline and can be used for MRI studies and volumetric-medical-imaging-modality studies such as ultrasound and CT studies. A main limitation of each of these approaches is that they are tailored and optimized for specific biomedical data modality and domain. While each data modality may need a specific treatment, there are commonalities across the different data modalities. An approach that can provide improved results across multiple modalities can shorten the development time for future predictive models. UniMiSS, a pyramid U-like Medical Transformer devised by Xie Y., et al., has been proposed to tackle this gap by utilizing multi-modal unlabeled medical images in a self-supervised manner. UniMiSS surpassed a diverse set of strong self-supervised approaches in a variety of medical-imaging learning tasks with different data modalities. (Caron, M. et al. Emerging properties in self-supervised vision transformers. arXiv 9650-9660 (2021); Zhou, H.-Y., et al., Preservational Learning improves self-supervised medical image models by reconstructing diverse contexts. arXiv 3499-3509 (2021); Xie, Y., et al., PGL: Prior-Guided Local Self-supervised Learning for 3D Medical Image Segmentation. arXiv (2020); Chen, X., et al., Improved Baselines with Momentum Contrastive Learning. arXiv (2020); Chen, X., et al., An Empirical Study of Training Self-Supervised Vision Transformers. arXiv (2021); the disclosures of which are incorporated by reference.) However, with respect to volumetric imaging, it was tested on a single classification problem in a single imaging modality (CT) while including this same imaging modality in its pre-training, and regression was not addressed. Thus, the full utility of transfer learning has yet to be attained across different modalities of volumetric-medical-imaging technologies.

Systems and methods described herein implement the slice integration by vision transformers (SLIViT) processes that use a 2D-based deep-learning heuristic framework that leverage deep vision modules with transfer learning to accurately detect disease-related risk factors in various volumetric biomedical imaging. Several embodiments combine a 2D ConvNeXt-based feature-map extractor and a vision transformer (ViT) module together with cross-dimension and cross-domain (such as, but not limited to, imaging modality, organ, and pathology) transfer learning. The 2D-based feature-map extractor allows leveraging prior 2D biomedical and/or non-biomedical vision knowledge when extracting information from a given volume in a variety of medical-imaging modalities. The ViT module allows to integrate the extracted information across the 2D frames of the volume.

The SLIViT processes can be applied in various medical domains including (but not limited to) retinal-disease risk biomarkers diagnosis in 3D OCT scans, pulmonary nodule-malignancy screening in 3D CT scans, cardiac function in echocardiogram videos, and hepatic-disease severity assessment from 3D MRI scans. SLIViT can attain improved performance compared to generic baselines and domain-specific models. In some embodiments, the architecture and hyperparameters stay invariant across tasks and/or data modalities, where SLIViT provides improved performance results across data modalities with neither tailoring the architecture nor optimizing hyperparameters per (task or) data modality. The performance of the SLIViT processes is comparable to clinical specialists' manual annotation, which can shorten the annotation time by a factor of at least 5,000. SLIViT can be used to save resources, reduce the burden on clinicians, and expedite ongoing research. Several embodiments show that SLIViT is robust to frame permutation, showing that (1) it can reconstruct long-range dependencies of the volume's depth dimension (that are likely ignored when the volume is tiled); and (2) it could be applied to datasets in which the slice order (within a volume) is not recorded. SLIViT does not require task-specific hyperparameter tuning and is relatively memory-thrifty, and thus can be effectively trained using standard hardware in reasonable time. SLIViT can facilitate generalizability, reproducibility, and applicability by a broader community of researchers to their datasets.

SLIViT can be applied to various 3D data modalities including (but not limited to) OCT scans, MRI scans, ultrasound videos, and computed tomography (CT) scans. SLIVit tackles various tasks including (but not limited to) five classification tasks (in OCT and ultrasound) and two regression problems (ultrasound and MRI). SLIViT can be used to classify OCT biomarkers including (but not limited to) drusen volume (DV), intraretinal hyperreflective foci (IHRF), subretinal drusen deposits (SDD), and hyporeflective drusen core (hDC), accurately and efficiently. In several embodiments, SLViT can be used to analyze the ejection fraction (EF) in echocardiogram videos. The EF is a key metric of cardiac function as it measures how well the heart's left ventricle is pumping blood. Low EF measurements (less than about 0.5) can indicate cardiomyopathy or other heart problems. If the EF labels (less than or equal to about 50%) are binarized and SLIViT is trained on the echocardiograms for cardiomyopathy binary classification, a 0.905 ROC AUC can be achieved. In some embodiments, SLIViT analyzes hepatic proton density fat fraction (PDFF) level, and/or proton density fat fraction in MRI images. SLIViT shows better performance than 3D Resnet and CNN methods (performance improved by about 10% to about 40%) and equivalence to clinical specialists' assessment performance with shorter time.

SLIViT combines two deep-vision architectures: a ConvNeXt backbone module that extracts feature maps (one per 2D slice), and a vision transformer module that integrates the feature maps into a single diagnosis prediction. In several embodiments, the backbone of SLIVit can be initialized by pre-trained weights. These weights can be obtained by training a 2D ConvNeXt (T variant) first on image databases such as (but not limited to) ImageNet and then on an independent B-scans dataset to classify retinal disease coarse risk factors. These pre-trained weights may allow SLIViT to excel in a variety of learning tasks especially when a small (less than or equal to a few hundreds of samples) training dataset is available. The backbone can be initialized with the same pre-trained weights and only fine-tuned according to the dataset and task in question. An underlining hypothesis is established that the basic features that are extracted from B-scans when learning one classification task could serve as a training starting point for data modalities such as 3D OCT, 3D CT, 3D ultrasound, and 3D MRI, as they share a basic set of features. In order to cope with volumetric data, which is essentially an array of 2D images, SLIViT tiles the 2D images into one elongated 2D image, keeping its width constant, such that it conforms with the input dimension expected by the 2D-based backbone. Once the feature maps are extracted, they can be comprehensively aggregated using a downstream vision transformer (with trainable positional embeddings) that aims to reconstruct the spatial signal that is lost when the volume is tiled into an elongated 2D image. Each original slice of the volume is being embedded into a single feature map.

SLIViT processes in accordance with many embodiments have permutation invariant properties. In the shuffling tests, the original frames order within each volume of a dataset (ultrasound dataset and/or MRI dataset) can be documented at the beginning. Then 100 random shuffled copies of the dataset can be generated, i.e., each volume is randomly shuffled in each shuffled copy. For each copy, a different data split from the one used in the Internal Test Experiment (to rule out split-dependent overperformance) can be used. 100 SLIViT models (one with each shuffled dataset) can be trained, and the performance of each model on the corresponding test set can be measured. The variance of the (consolidated) performance of all 100 models is higher than the performance of a single model. The performance of the original model can be captured within the consolidated performance confidence interval of the 100 shuffling experiments.

### Analyzing Volumetric Biomedical Imaging Data

Several embodiments implement SLIViT for automatic annotation of medical features in three-dimensional biomedical images. SLIViT preprocesses 3D volumes into 2D images and then combines two deep vision architectures: (1) a ConvNeXt backbone module that extracts feature maps for the slices (i.e., 2D frames of a volume), and (2) a ViT module that integrates the slices feature maps into a single diagnosis prediction.

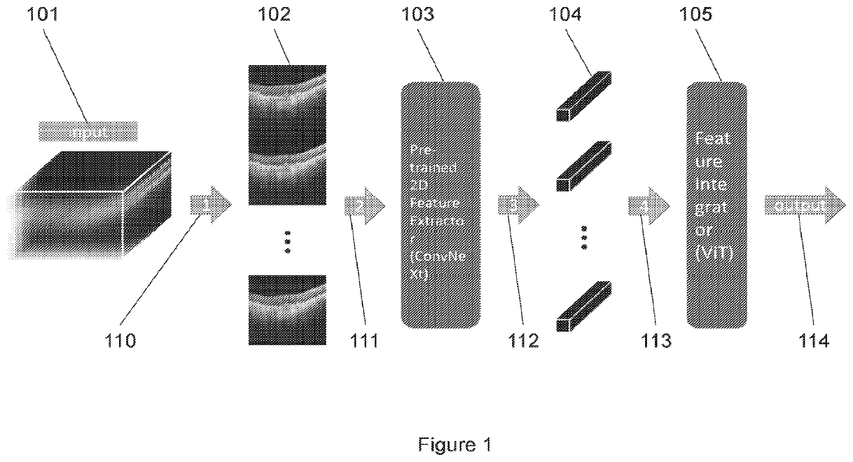

FIG. 1 illustrates a schematic of SLIViT process in accordance with an embodiment. The input 101 of SLIViT can be a 3D volume of N frames of size H×W. The frames of the volume can be resized and vertically tiled (110) into an “elongated image” 102. To cope with 3D volumetric data, several embodiments treat each volume as a set of slices. Each original slice of the volume can be embedded into a single feature map. SLIViT reduces memory overhead and accelerates the processing time by tiling the 2D images into a single elongated 2D image (rather than a set of separate images), such that it conforms with the input dimension expected by the 2D-based feature-map extractor 103.

The elongated image 102 can be fed (111) into a ConvNeXt-based feature extractor 103 that is pre-trained on both natural and medical 2D labeled images. The feature map extractor can be initialized by pre-trained weights. These weights can be obtained by pre-training a 2D ConvNeXt (T variant) first on natural image datasets such as ImageNet. The weights can then be trained on an independent 2D OCT B-scan dataset and labeled with retinal-disease coarse risk factors. These pre-trained weights, that are used for initialization on each of the experiments, allow SLIViT to improve the performance in a variety of learning tasks especially when a small (few hundreds of samples) training dataset is available. The basic features that are extracted from 2D biomedical imaging when learning one task could serve as an improved training starting point for 3D biomedical imaging learning tasks involving different organs and different imaging modality (such as (but not limited to) 3D OCT scans, ultrasound videos, 3D CT scans, and 3D MRI scans) as they share basic sets of features.

N feature maps 104 can be extracted (112) (each corresponding to an original frame). The feature maps 104 can be fed (113) into a ViT-based feature integrator 105 followed by a fully connected layer that outputs (114) the prediction for the task in question. Once the feature maps 104 are extracted, they are paired with (trainable) positional embeddings and comprehensively aggregated using a downstream ViT module 105. SLIViT's ViT module together with (trainable) positional embeddings allow to preserve the long-range dependencies across the depth dimension if needed. The ViT's attention mechanism implicitly eliminates the necessity for image registration preprocessing.

In some embodiments, SLIViT are tested on six datasets of four different volumetric medical imaging data modalities (OCT, CT, ultrasound, and MRI) with a limited number of annotated samples, tackling a variety of clinical-feature learning tasks (including both classification and regression). Several embodiments evaluate the diagnosis performance of ocular disease high-risk factors in OCT scans and malignant pulmonary nodules in CT scans and measure it by both the receiver operating characteristic (ROC) area under the curve (AUC) and precision-recall (PR) AUC. The ultrasound and MRI tests in accordance with certain embodiments compare the R2 of the models' predictions vs. ground truth in (respectively) cardiac function analysis and in hepatic fat level imputation. In each data modality, SLIViT performance is compared with a diverse set of up to six baselines, including domain-specific and generic (fully-supervised-based and self-supervised-based) methods. SLIViT processes show consistent and improved performance across domains.

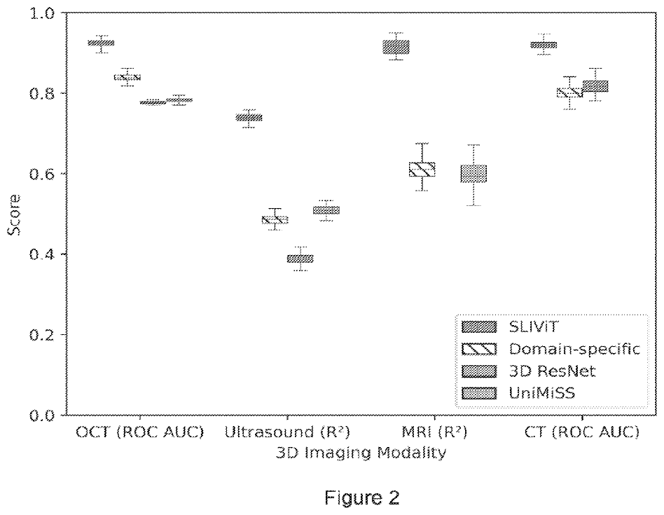

FIG. 2 illustrates performance overview of the SLIViT processes in accordance with an embodiment. FIG. 2 shows the performance scores in one classification task (with two different metrics) of eye disease biomarker diagnosis in volumetric-OCT scans and two regression tasks of (1) heart function analysis in ultrasound videos and (2) liver fat levels imputation in volumetric MRI scans. Domain-specific methods (hatched) include SLIVER-net, EchoNet, and 3D ResNet, for OCT, ultrasound, and MRI, respectively. The general cross-modality benchmarking used are 3D ResNet (green) and UniMiSS (brown) which are fully-supervised-based and self-supervised-based, respectively. Box plot whiskers represent a 90% CI. SLIVER-net, EchoNet, 3D ResNet, and UniMiSS methods are described in the references. (See, e.g., Tran, D. et al. A Closer Look at Spatiotemporal Convolutions for Action Recognition. arXiv (2017); Arnab, A. et al. ViViT: A Video Vision Transformer. arXiv (2021); Rakocz, N. et al. NPJ Digit Med 4, 44 (2021); Gupta, U. et al. Transferring Models Trained on Natural Images to 3D MRI via Position Encoded Slice Models. ArXiv (2023); Azizi, S. et al. Nat Biomed Eng 7, 756-779 (2023); Xie, Y., Zhang, J., Xia, Y. & Wu, Q. UniMiSS: Universal Medical Self-Supervised Learning via Breaking Dimensionality Barrier. arXiv (2021); the disclosures of which are incorporated by reference.)

### Detecting Ocular Disease High-Risk Factors Using 3D OCT Scans

SLIViT's performance is compared against trained SLIVER-net, 3D ResNet, 3D ViT, and UniMiSS models, on the Houston Dataset which includes 691 OCT B-scan volumes of different individuals. OCT B-scan volume data are collected from independent individuals affected in at least one eye by dry AMD, a leading cause of irreversible central visual impairment. Each OCT volume has four different binary labels of AMD high-risk biomarkers procured by a senior retina specialist-drusen volume larger than 0.03 mm³ (DV), intraretinal hyperreflective foci (IHRF), subretinal drusen deposits (SDD), and hyporeflective drusen cores (hDC). The dataset can be randomly split into train, validation, and test sets of sizes 483 (70%), 104 (15%), and 104 (15%), respectively. Four different SLIViT models (one per binary label) are trained. The ROC AUC and PR AUC scores (the latter is also known as average precision or average positive predictive value) are used for performance evaluation. The models are trained (using less than 600 volumes) and tested on the same split.

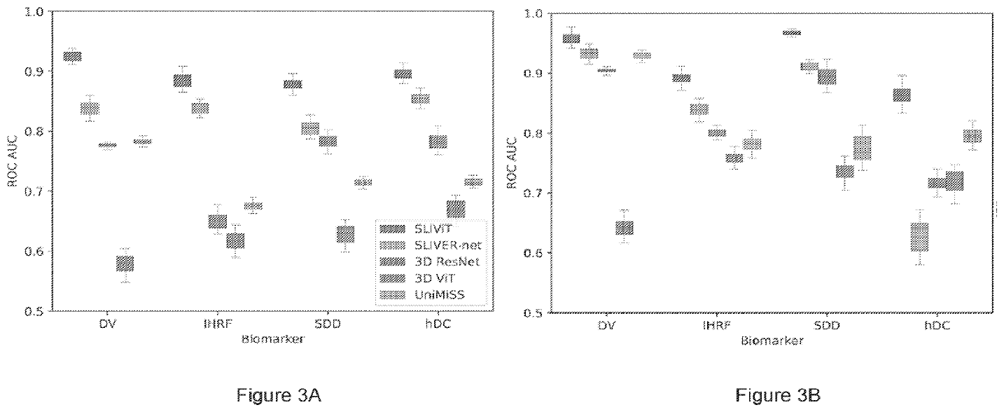

FIGS. 3A and 3B illustrate ROC AUC performance comparison of five models in accordance with an embodiment. The ROC AUC scores of SLIViT (blue), SLIVER-net (orange), 3D ResNet (green), 3D ViT (red), and UniMiSS (brown) on four single-task classification problems of AMD high-risk factors in two independent volumetric-OCT datasets are shown. The expected performance of a naive classifier is 0.5. FIG. 3A shows the performance when trained and tested on the Houston Dataset. FIG. 3B shows the performance when trained on the Houston Dataset and tested on the SLIVER-net Dataset. Box plot whiskers represent a 90% CI.

FIG. 3A shows the ROC AUC performance comparison in four independent AMD-biomarker classification tasks when trained on less than 700 OCT volumes. In all four biomarkers, SLIViT outperforms the other approaches in both evaluation metrics. For example, in the DV classification task (also shown as the OCT experiment in FIG. 2) SLIViT (ROC AUC=0.924; CI [0.909, 0.938]) is better compared to the SLIVER-net method (ROC AUC=0.838; CI [0.813, 0.86]; paired t-test p-value<0.001). In terms of average precision of the DV classification, SLIViT (PR AUC=0.914; CI [0.898, 0.928]) is better compared to the 3D ResNet method (PR AUC=0.759; CI [0.748, 0.769]; paired t-test p-value<0.001). Since the biomarkers are structural, their identification requires aggregation of three-dimensional information. Thus, the ability of SLIViT to successfully identify these biomarkers suggests that it adequately captures a three-dimensional signal within a given volume.

SLIViT is also tested with the SLIVER-net dataset as shown in FIG. 3B. In this task, SLIVER-net should have an advantage as it is optimized for this dataset. The SLIVER-net dataset includes about one thousand OCT scans (imaged from independent individuals in an Amish population) collected from three different clinical centers. SLIViT, SLIVER-net, 3D ResNet, 3D ViT, and UniMiSS, are trained using the 691 Houston Dataset volumes. The SLIVER-net Dataset is used as the test set. For some biomarker classification tasks, the relative improvement of SLIViT compared to SLIVER-net is reduced. SLIViT still outperforms the other approaches, in any of the four AMD-biomarker classification tasks.

Table 1 summarizes the average classification performance ROC AUC scores of SLIViT, SLIVER-net, 3D ResNet, 3D ViT, and UniMiSS trained on less than 700 OCT volumes. Table 1 includes the performance raw numbers underlying ROC AUC of the AMD high-risk biomarker prediction experiments. The numbers in the square brackets represent the corresponding 90% CI.

TABLE 1
ROC AUC Scores

| Test dataset | Method | DV | IHRF | SDD | hDC |
|---|---|---|---|---|---|
| Houston | SLIViT | .924 | .883 | .877 | .89 |
| | | [.909, .938] | [.86, .906] | [.855, .893] | [.877, .916] |
| | SLIVER-net | .838 | .837 | .805 | .854 |
| | | [.813, .86] | [.82, .855] | [.78, .827] | [.836, .869] |
| | 3D ResNet | .777 | .655 | .783 | .782 |
| | | [.769, .783] | [.625, .682] | [.762, .806] | [.757, .805] |
| | 3D ViT | .576 | .617 | .629 | .667 |
| | | [.547, .605] | [.583, .651] | [.598, .66] | [.63, .703] |
| | UniMISS | .783 | .675 | .714 | .715 |
| | | [.771, .793] | [.66, .69] | [.701, .726] | [.7, .729] |
| SLIVER-net | SLIViT | .958 | .891 | .967 | .863 |
| | | [.941, .975] | [.873, .909] | [.959, .973] | [.839, .892] |
| | SLIVER-net | .933 | .839 | .911 | .625 |
| | | [.919, .95] | [.817, .86] | [.9, .922] | [.576, .676] |
| | 3D ResNet | .904 | .8 | .895 | .716 |
| | | [.891, .911] | [.788, .813] | [.865, .925] | [.689, .737] |
| | 3D ViT | .642 | .758 | .735 | .718 |
| | | [.611, .674] | [.737, .78] | [.7, .77] | [.677, .758] |
| | UniMISS | .929 | .781 | .774 | .795 |
| | | [.915, .939] | [.753, .808] | [.723, .825] | [.765, .825] |

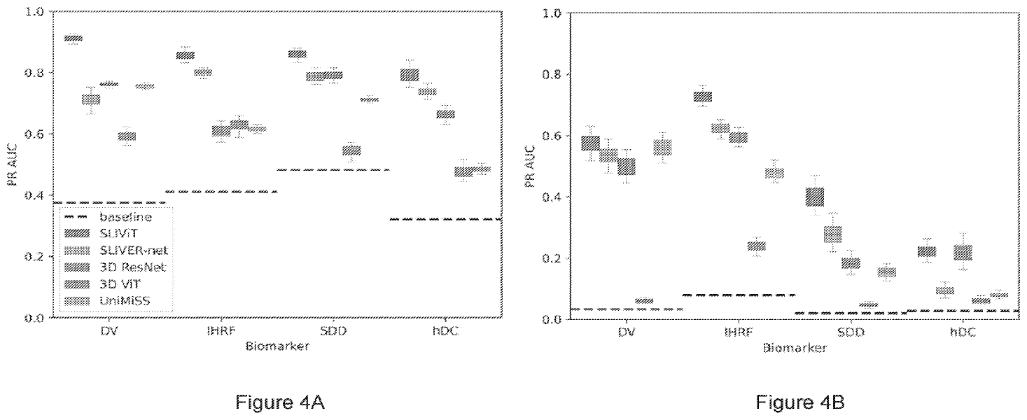

FIGS. 4A and 4B illustrate PR AUC performance comparison of five models in accordance with an embodiment. FIGS. 4A and 4B show the PR AUC performance comparison in four independent AMD-biomarker classification tasks when trained on less than 700 OCT volumes. The PR AUC scores are shown as an alternative scoring metric for the experiment shown in FIG. 3. The dashed lines represent the corresponding biomarker's positive-label prevalence, which is the expected PR AUC score of a naive classifier. FIG. 4A shows the performance when trained and tested on the Houston Dataset. FIG. 4B shows the performance when trained on the Houston Dataset and tested on the SLIVER-net Dataset. Box plot whiskers represent a 90% CI.

Table 2 summarizes the average classification performance PR AUC scores of SLIViT, SLIVER-net, 3D ResNet, 3D ViT, and UniMiSS trained on less than 700 OCT volumes. Table 2 includes the performance raw numbers underlying PR AUC of the AMD high-risk biomarker prediction experiments. The numbers in the square brackets represent the corresponding 90% CI.

TABLE 2
PR AUC Scores

| Test dataset | Method | DV | IHRF | SDD | hDC |
|---|---|---|---|---|---|
| Houston | SLIViT | .914 | .852 | .855 | .795 |
| | | [.898, .928] | [.826, .875] | [.831, .879] | [.747, .838] |
| | SLIVER-net | .708 | .799 | .785 | .74 |
| | | [.676, .744] | [.778, .817] | [.752, .816] | [.716, .76] |
| | 3D ResNet | .759 | .619 | .791 | .669 |
| | | [.748, .769] | [.584, .647] | [.77, .815] | [.622, .697] |
| | 3D ViT | .589 | .627 | .54 | .479 |
| | | [.551, .628] | [.584, .67] | [.494, .586] | [.428, .529] |
| | UniMiSS | .755 | .616 | .711 | .484 |
| | | [.742, .769] | [.598, .634] | [.696, .726] | [.462, .506] |
| SLIVER-net | SLIViT | .575 | .728 | .399 | .222 |
| | | [.517, .63] | [.696, .763] | [.341, .469] | [.184, .263] |
| | SLIVER-net | .535 | .621 | .278 | .093 |
| | | [.47, .588] | [.588, .653] | [.221, .345] | [.07, .122] |
| | 3D ResNet | .497 | .593 | .183 | .219 |
| | | [.444, .553] | [.563, .626] | [.147, .225] | [.162, .282] |
| | 3D ViT | .06 | .238 | .046 | .061 |
| | | [.046, .074] | [.199, .276] | [.032, .061] | [.042, .08] |
| | UniMiSS | .56 | .48 | .153 | .08 |
| | | [.497, .623] | [.431, .528] | [.114, .191] | [.061, .099] |

### Analyzing Cardiac Function Using Ultrasound Videos

To evaluate SLIViT's generalizability, several embodiments test the algorithms on different 3D data modalities. The EchoNet-Dynamic Dataset contains 10,030 standard apical four-chamber view ultrasound videos (echocardiograms) obtained from unrelated individuals, each associated with a continuous number representing the corresponding ejection fraction (EF) measured in a clinical setting. The EF is measured by tracing the chamber volume of the left ventricle in the end-systole and end-diastole. The EF can be a key metric of cardiac function as it measures how well the heart's left ventricle is pumping blood. Low EF measurements (less than about 0.5) can indicate cardiomyopathy or other heart problems. SLIViT's can be used to predict cardiomyopathy as a binary classification task. The EF measurements can be binarized accordingly (greater than or equal to about 0.5 is normal) using the original EchoNet-Dynamic Dataset split. SLIViT and 3D ResNet can be trained.

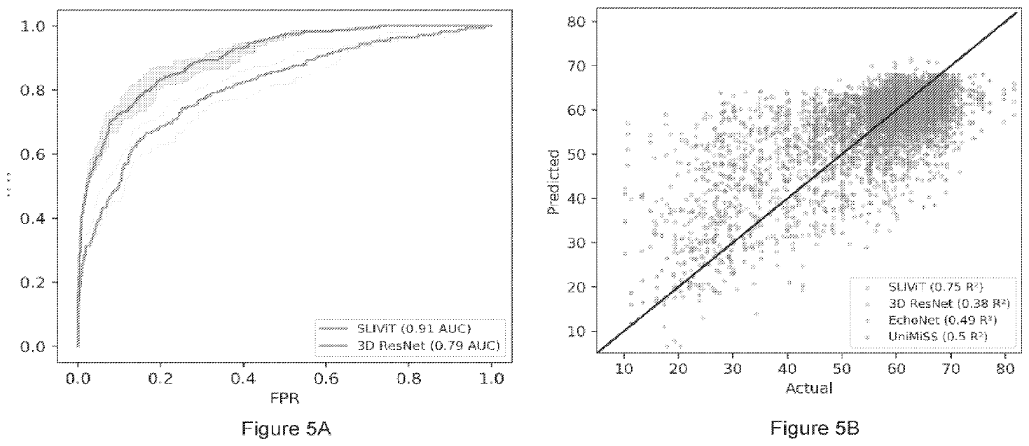

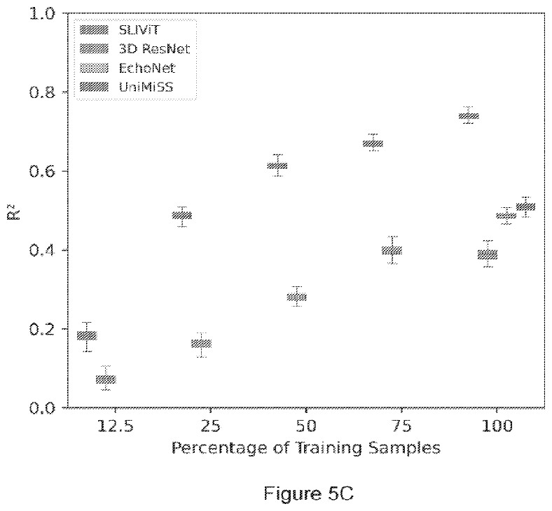

FIGS. 5A through 5C illustrate performance comparison on cardiac function prediction tasks using echocardiograms in accordance with an embodiment. Box plot whiskers represent a 90% CI. When SLIViT is trained on 25% (n=1,866) of the original training set, it can achieve similar accuracy as the other examined methods when trained on 100% (n=7,465) of the training set. FIG. 5A shows ROC curves of cardiomyopathy prediction (EF<0.5). SLIViT achieves 0.913 ROC AUC (CI [0.901, 0.928]) and overperforms 3D ResNet with 0.793 ROC AUC (CI [0.772, 0.814]) (paired t-test p-value<0.001).

SLIViT is tested in a regression task. FIG. 5B shows predicted vs. actual EF levels for three different models trained on the original training set (solid line represents the y=x line). EchoNet achieves R2=0.489; CI [0.434, 0.526]. Trained with the same dataset split, SLIViT achieves an improvement of 0.75 R2 (CI [0.706, 0.781]; paired t-test p-value<0.001). 3D ResNet and UniMiSS underperform SLIViT with 0.384 (CI [0.364, 0.413]) and 0.502 (CI [0.487, 0.531]) R2, respectively. Several embodiments examine (1) a factorized spatiotemporal ResNet architecture (R(2+1)D, in contrast to the 3D-filter-based R3D ResNet that is known to capture both spatial and temporal features from video frames, and (2) 3D ViT. Both methods perform below par compared to the other abovementioned benchmarks (R2=−0.081; CI [−0.106, −0.056] and R2=0.333; CI [0.27, 0.396], respectively).

Several embodiments examine the dynamics of the training set size and SLIViT's performance in predicting the EF of a given echocardiogram. FIG. 5C shows R2 performance of heart EF prediction using different percentages of the original training dataset. Some embodiments randomly sample size-decreasing subsets from the original training set and train a SLIViT model per subset. Compared to other examined methods trained on the original training set (n=7,465), when SLIViT uses the 25% subset (n=1,866) its performance (R2=0.487; CI [0.466, 0.507]) is better than R3D, R(2+1)D, and 3D ViT (paired t-test p-value<0.001); on par with EchoNet (paired t-test p-value>0.579); and lower than UniMiSS (paired t-test p-value<0.001). When SLIViT uses the 50% subset, it outperforms all other benchmarked methods (R2=0.614; CI [0.594, 0.634]; paired t-test p-value<0.001). These observations substantiate SLiT's ability to appropriately learn spatiotemporal features using a sparsely labeled dataset.

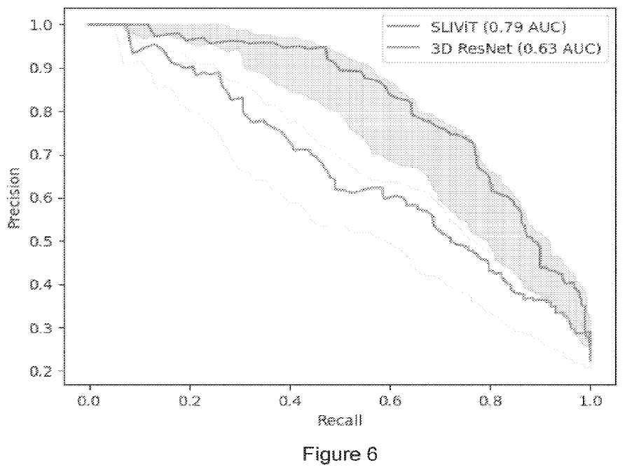

FIG. 6 illustrates performance comparison of a cardiomyopathy binary classification task on echocardiograms in accordance with an embodiment. FIG. 6 shows the PR curves yielded by modeling SLIViT and 3D ResNet to classify cardiomyopathy. The shaded areas represent a 90% CI.

### Predictinq Hepatic Fat Levels in 3D MRI Scans

In several embodiments, SLIViT is implemented in 3D MRI data. UK Biobank Dataset containing 3D hepatic MRI scans and a corresponding measurement for hepatic proton density fat fraction (PDFF) level is used. The PDFF measurement provides an accurate estimation of hepatic fat levels and it is proposed as a non-invasive method to limit unnecessary hepatic biopsies. An accurate quantitative measurement of fat can be important in improving the diagnosis of various fatty-liver and diabetes-related diseases. The unlabeled scans are removed, and the rest of the dataset is preprocessed to contain a single scan per individual. SLIViT is compared to 3D ResNet and UniMiSS. The dataset is randomly split, and the models are trained to measure PDFF levels of a given 3D MRI. SLIViT can achieve 0.916 R2 (CI [0.879, 0.952]) and outperforms both 3D ResNet and UniMiSS that obtain 0.611 (CI [0.566, 0.644]) and 0.599 (CI [0.531, 0.667]) R2, respectively (paired t-test p-value<0.001; See MRI experiment in FIG. 2). The performance of 3D ViT and a 2D-Slice-CNN-based architecture is evaluated, and they both show poor performance compared to the above benchmarks (R2=0.18 (CI [0.145, 0.214]) and −0.130 (CI [−0.111, −0.148]), respectively).

### Classifying Nodule Malignancy in 3D CT Scans

In many embodiments, the generalizability of SLIViT is shown with 3D CT data. The NoduleMNIST3D Dataset containing 3D thoracic CT scans, each (binary) labeled for nodule malignancy, is used. In the United States, more than a million patients are diagnosed with pulmonary nodules each year, and these nodules are observed in roughly 30% of thoracic CT scans. As in other biomedical imaging domains, the scan screening is subjective and depends on the clinical specialist's experience (e.g., small nodules may be missed. Efficient and accurate assessment could hasten malignant pulmonary nodule treatment and reduce unnecessary testing when benign. Using the dataset's predefined split, SLIViT, 3D ResNet, and UniMiSS are trained and the results are compared on the test set (see CT experiment in FIG. 2). UniMiSS is pre-trained on more than 5,000 3D thoracic CT scans (i.e., more than 4× larger than the NoduleMNIST3D training set), and thus, has a potential advantage. Yet, SLIViT obtains 0.926 ROC AUC (CI [0.904, 0.947]) and 0.785 PR AUC (CI [0.758, 0.837) and significantly overperforms (paired t-test p-value<0.001) both UniMiSS with 0.8 ROC AUC (CI [0.765, 0.836]) and 0.627 PR AUC (CI [0.568, 0.685]), 3D ResNet with 0.821 ROC AUC (CI [0.776, 0.857]) and 0.619 PR AUC (CI [0.508, 0.718]). The performance of 3D ViT is evaluated and obtains 0.873 ROC AUC (CI [0.825, 0.914]) and 0.713 PR AUC (CI [0.627, 0.792]). SLIViT performs better than other methods (paired t-test p-value<0.TODO).

### Comparable Quality as Clinical Specialists

Several embodiments show potential utility of automating the detection of AMD high-risk biomarkers with SLIViT with the Pasadena Dataset, a 3D OCT dataset containing 205 3D OCT volumes of (205) independent individuals. The ground truth for this dataset is obtained by three senior retina specialists. Seven junior clinicians independently annotate each of the OCT volumes in this dataset for four AMD high-risk biomarkers: DV, IHRF, SDD, and hDC. The SLIViT model that is trained on the 691 Houston dataset also annotates these volumes.

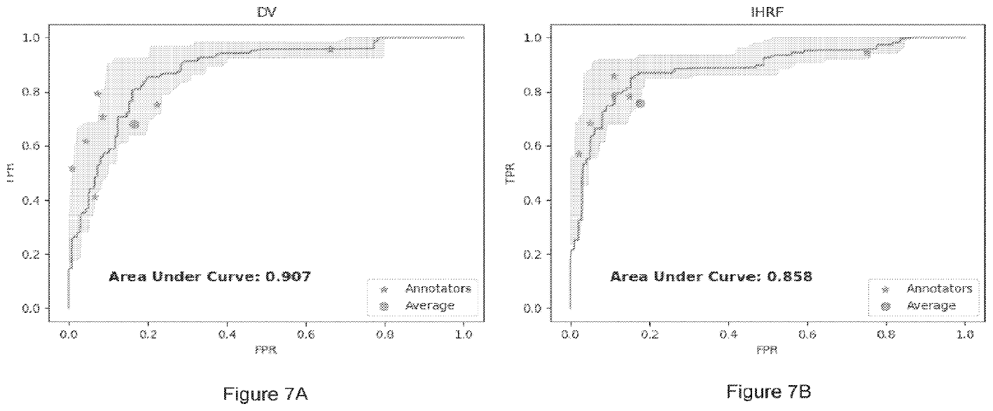

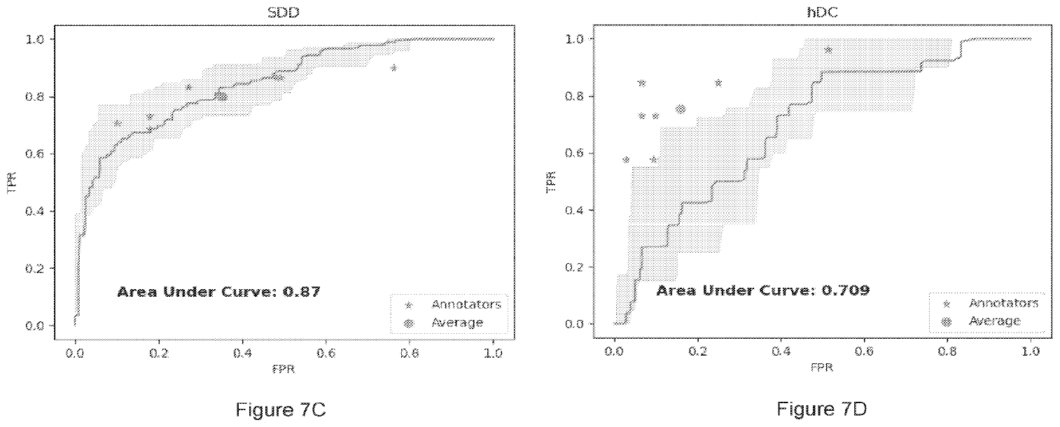

FIGS. 7A through 7D illustrate SLIViT's ROC curve compared to junior clinical retina specialists' assessment in accordance with an embodiment. FIGS. 7A through 7D show the ROC curves of SLIViT trained to predict four AMD high-risk biomarkers (DV, IHRF, SDD, and hDC) using less than 700 OCT volumes (Houston Dataset) and tested on an independent dataset (Pasadena Dataset). The shaded area represents a 90% CI for SLIViT's performance. The red dot represents the specialists' average performance. The green asterisks correspond to the retina specialists' assessments. Two of the clinical specialists obtain the same performance score for IHRF classification.

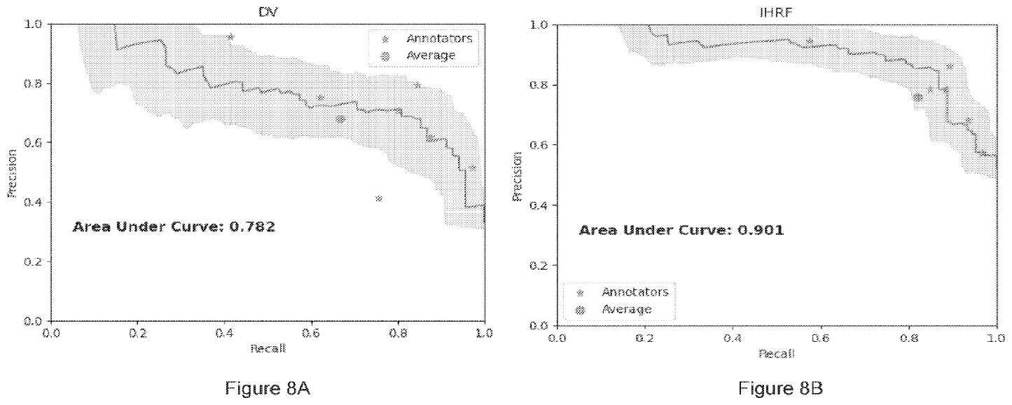

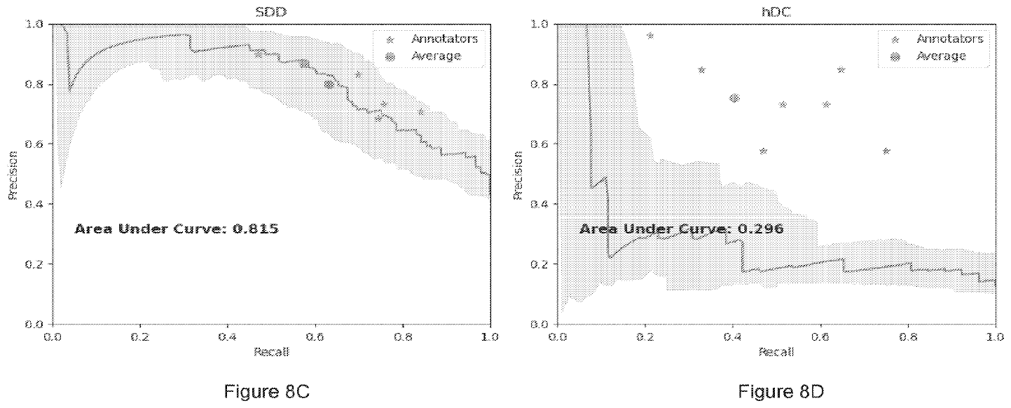

FIGS. 8A through 8D illustrate SLIViT's PR performance compared to junior clinical retina specialists' assessment in accordance with an embodiment. FIGS. 8A through 8D show the PR curves of SLViT trained to predict four AMD high-risk biomarkers (DV, IHRF, SDD, and hDC) using less than 700 OCT volumes (Houston Dataset) and tested on an independent dataset (Pasadena Dataset). The shaded area represents a 90% CI for SLViT's performance. The red dot represents the specialists' average performance. The green asterisks correspond to the retina specialists' assessments. Two of the clinical specialists obtain the same performance score for IHRF classification.

FIGS. 7A through 8D summarize respectively the true positive rate (TPR; also known as recall) vs. false positive rate (FPR; also known as false alarm rate) and the positive predictive value (PPV; also known as precision) vs. recall of SLIViT and the seven junior clinicians over the Pasadena Dataset. Clinicians typically can reach comparable performance but have to invest 5,000-fold more time to do so (on average, it takes about 17 working hours net for each clinician to procure the annotations while SLIViT completes the job in under 12 seconds). SLIViT obtains lower performance in the hDC classification task compared to the other biomarker classification tasks. A possible reason can be the absence of a universal consensus on the clinical definition of hDC. This feature has a highest senior specialists' annotation discordance among the four biomarkers, suggesting that it is harder to distinguish between cases.

### Robustness in Volume Frame Permutation

Several embodiments show SLIViT's robustness to changes in the order of the frames encoding a volume. Some embodiments generate about 100 copies of the Houston Dataset and randomly shuffle each volume (in each of these 100 copies). Some embodiments use the same split to train 100 SLIViT models (one per shuffled copy; henceforth “shuffled models”) and one model on the Houston Dataset using the original order (henceforth “original model”) to classify the structural AMD high-risk factors: DV, IHRF, SDD, and hDC.

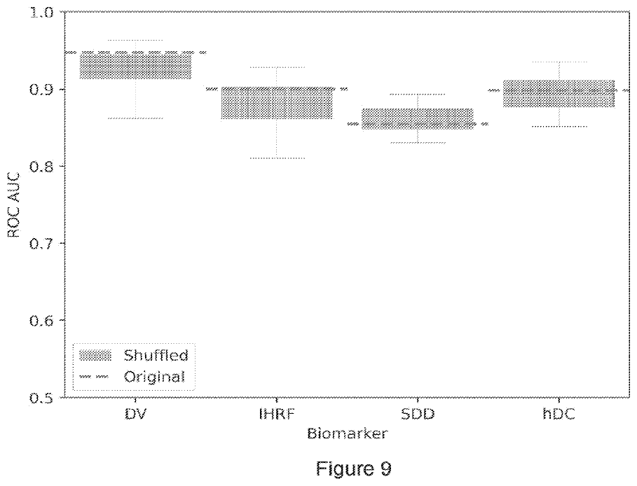

FIG. 9 illustrates SLIViT's performance in a volumetric-OCT frame-permutation experiment in accordance with an embodiment. FIG. 9 shows the ROC AUC scores distribution of 100 shuffled models trained on 100 different (shuffled) copies of a volumetric-OCT dataset. The expected performance of a naive classifier is about 0.5. Box plot whiskers extend to the 5th and the 95th percentiles of the 100 shuffled models' performance distribution. The dashed line represents the performance of a SLIViT model trained on the volumetric-OCT dataset using the original order of each volume. The performance ranks of the shuffled models compared to the original models' distribution are about 22, 34, 56, and 47 for DV, IHRF, SDD, and hDC, respectively.

FIG. 9 shows the average bootstrapped ROC AUC dispersion of the 101 models. The original model does not outperform the shuffled models. Compared to the 100 shuffled-models performance, the average rank of the original model across the four AMD biomarkers is about 40. This finding suggests that even if the original order is not documented, SLIViT's performance does not deteriorate. SLIViT can effectively aggregate information across slices, even when the order of slices is not maintained.

The frame-permutation invariance can be intriguing, especially when the examined biomarkers are structural. Some embodiments train a tweaked SLIViT model with one multihead attention layer (instead of five in the original version) in which non-immediately adjacent attention weights are set to zero. This tweaked version can pool information only from immediately adjacent frames in the volume. The tweaked SLIViT model is trained on the original dataset and evaluated the performance on the test set twice, once with frames in order (0.912 ROC AUC (CI [0.91, 0.919]) and 0.908 PR AUC (CI [0.901, 0.914])) and once with random frame shuffling (0.846 ROC AUC (CI [0.832, 0.862]) and 0.827 PR AUC (CI [0.805, 0.838]). The noticeable decline in the performance (0.066 and 0.081 in ROC and PR AUC scores, respectively) of the model when evaluated on the shuffled test set suggests that SLIViT's performance on this learning task relies on successfully pooling information from different frames.

### The Utility of Pre-Training

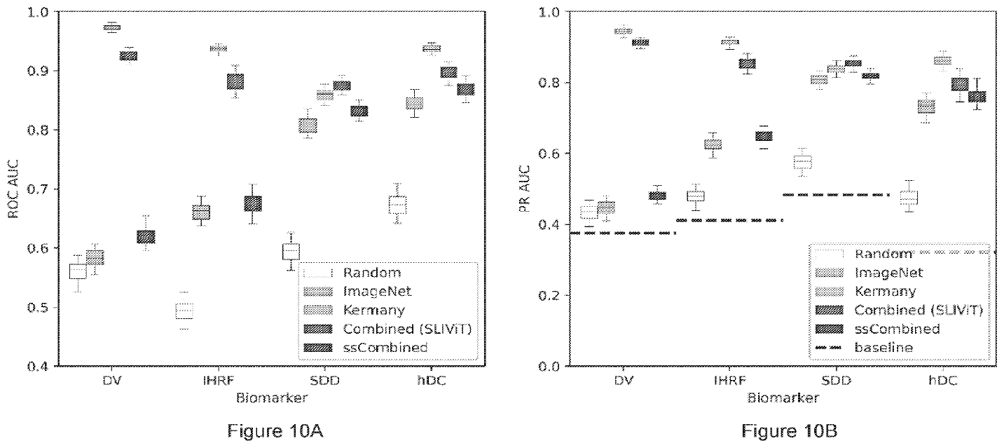

The utility of ImageNet pre-training (henceforth “ImageNet weights”) can be used in various biomedical-imaging learning tasks. However, transfer learning between unrelated domains remains controversial and commonalities across biomedical imaging data modalities may be counterintuitive. Several embodiments provide a pre-training ablation study across the different learning tasks to evaluate the benefit of the cross-modality and cross-dimensionality transfer learning and assess the contribution of different selections made for the pre-training step of SLIViT. FIGS. 10A and 10B illustrate pre-training ablation study for (volumetric) OCT-related downstream learning tasks in accordance with an embodiment. FIG. 10A shows the ROC AUC scores and FIG. 10B shows the PR AUC scores across different fine-tuned models for volumetric-OCT classification tasks initialized with six different sets of pre-trained weights. The expected ROC AUC score of a naive classifier is 0.5. Combined, the proposed SLIViT's initialization, is ImageNet weights initialization followed by supervised pre-training on the Kermany Dataset. ssCombined is an ImageNet weights initialization followed by self-supervised pre-training on an unlabeled version of the Kermany Dataset. The dashed lines represent the corresponding biomarker's positive-label prevalence, which is the expected PR AUC score of a naive classifier. Box plot whiskers represent a 90% CI.

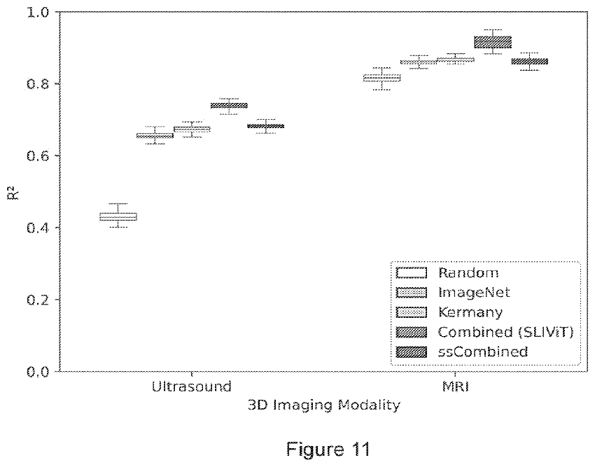

FIG. 11 illustrates pre-training ablation study for (volumetric) non-OCT-related downstream learning tasks in accordance with an embodiment. FIG. 11 shows the R2 scores for the volumetric ultrasound and MRI regression tasks initialized with six different sets of pre-trained weights. Combined, the proposed SLIViT's initialization, is ImageNet weights initialization followed by supervised pre-training on the Kermany Dataset. ssCombined is an ImageNet weights initialization followed by self-supervised pre-training on an unlabeled version of the Kermany dataset. Box plot whiskers represent a 90% CI.

FIGS. 10A through 11 compare four different initializations: random weights, ImageNet weights, random weights initialization followed by 2D OCT B-scans pre-training (henceforth “Kermany weights”), and ImageNet weights initialization followed by 2D OCT B-scans pre-training (henceforth “combined weights”). The combined weights is the original initialization approach for SLIViT. The results of this experiment indicate the following observations. First, using ImageNet weights can improve performance for the data modalities tested relative to random weights. Utilizing 2D OCT B-scans in pre-training (either Kermany weights relative to random weights or combined weights relative to ImageNet weights) can improve performance in downstream learning tasks. In the four OCT-related classification tasks, using Kermany weights (that is, without ImageNet) is a better approach and leads to better performance, even when compared to the combined approach (FIGS. 10A and 10B). Moreover, only pre-training strategies that leverage the 2D OCT B-scan dataset at full, i.e., Kermany weights and combined weights (FIGS. 10A and 10B) show consistent superior performance relative to all other tested benchmark methods (FIGS. 3A, 4A, and 10A). In ultrasound, MRI, and CT experiments, SLIViT can achieve superior performance relative to other benchmark methods tested, regardless of the pre-training strategy (FIGS. 2 and 11). This finding demonstrates the advantage of SLIViT's architecture for cross-modality volumetric-medical-imaging learning tasks.

### Self-Supervised Pre-Training

Self-supervised learning can be a useful approach in visual tasks, such as in the medical-imaging domain where procuring annotations is laborious and expensive. Some embodiments provide the utility of self-supervised-based pre-training approach on SLIViT using an unlabeled version of the 2D OCT B-scans dataset. The REMEDIS approach is tested, and the performance is robust to different self-supervised techniques. The REMEDIS default scheme is used, and SimCLR88 is used as the self-supervised technique. REMEDIS is originally shown to obtain remarkable performance when pre-trained even on much smaller (unlabeled) datasets than the 2D OCT B-scans dataset. Several embodiments provide that initializing SLIViT with the fully supervised pre-trained weights can outperform the self-supervised initialization in downstream learning tasks (paired t-test p-value<0.001; FIGS. 10A through 11). The same performance-superiority conclusion regarding the competitor benchmarks from the previous section held for the self-supervised-based version of SLIViT, implying its potential to harness unlabeled data when available.

In ultrasound and MRI experiments, SLIViT achieves better performance relative to other benchmarks tested, regardless of the pre-training strategy (FIGS. 2 and 11). This discovery further demonstrates the advantage of SLIViT's architecture for out-of-distribution volumetric-medical-imaging learning tasks. For the in-distribution medical imaging task, that is the (3D) OCT experiment, only pre-training strategies that leverage the 2D OCT B-scan dataset at full, i.e., Kermany weights and combined weights, show consistent better performance relative to other tested benchmark methods (left panels of FIGS. 3A, 4A, and 10A).

### 2D Biomedical-Imaging Pre-Training

Several embodiments compare the utility of different 2D biomedical-imaging-data modalities. Several embodiments define Kermany weights, obtained by random weights initialization followed by 2D OCT (using the Kermany Dataset) pre-training of SLIViT's feature-map extractor backbone. Several embodiments define “Organ weights” and “Chest weights”, obtained by random weights initialization followed by (respectively) 2D CT (using Organ{A,C,S}MNIST) and 2D X-ray (using ChestMNIST) pre-training. Some embodiments use Kermany weights, Organ weights, and Chest weights to conduct the following two experiments. Certain embodiments examine the similarities between the representations learned by biomedical-weights-initialized SLIViT backbones (without downstream-task-specific fine-tuning). To this end, three backbones are initialized with Kermany weights, Organ weights, and Chest weights (henceforth “biomedical backbones”), and two additional baselines, one with ImageNet weights and another with random weights (henceforth “ImageNet backbone” and “random backbone”, respectively). The NoduleMNIST3D Dataset is taken and induced its projections using these five backbones and measured the central kernel alignment (CKA) similarity index between pairs of projections. Consider two CKA-score distributions for the comparison—the distribution of the top-5% CKA scores (henceforth “top-5% distribution”), which are likely to be enriched with informative features, and the distribution of the overall CKA scores (henceforth “overall distribution”). First, each of the three biomedical backbones is more similar to the other two biomedical backbones than to the random and the ImageNet backbones (t-test p-value<0.001). This finding holds when the corresponding top-5% distributions and the overall distributions are compared. When comparing the overall distribution of two biomedical backbones to the top 5% distribution of a biomedical backbone and a non-medical backbone, robust results can be observed. For example, the overall distribution for the Kermany backbone and each of the other two biomedical backbones is comparable to the top 5% distribution of Kermany and ImageNet backbones (t-test p-value>0.05), and higher compared to the top 5% distribution of Kermany and random backbones (t-test p-value<0.001). The same is observed for the other two biomedical backbones. These findings confirm the hypothesis that different data modalities share a basic set of features.

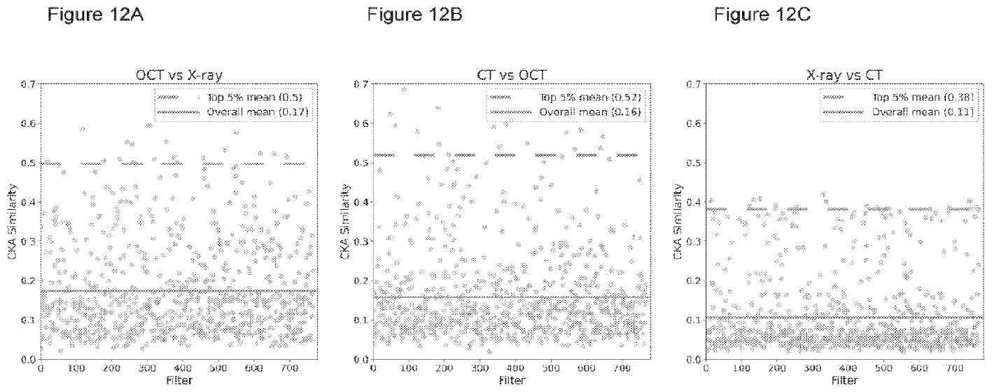

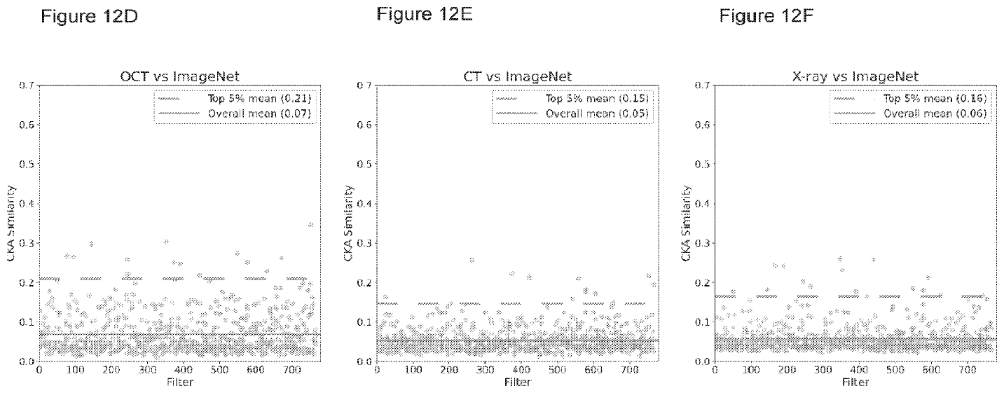

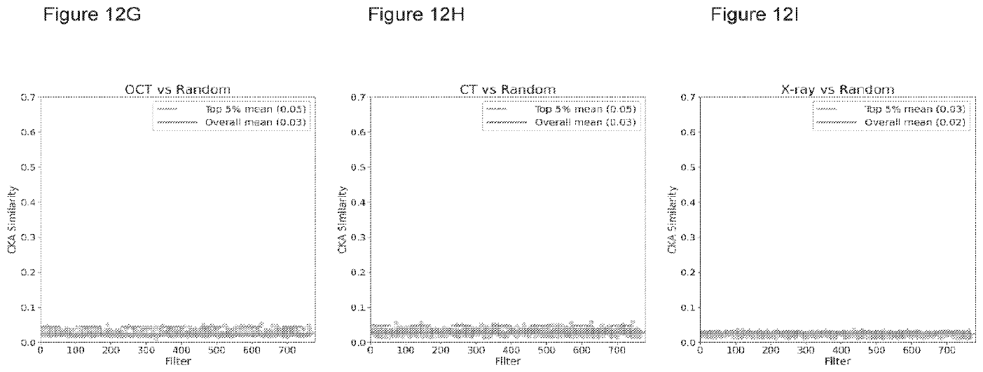

FIGS. 12A through 12I illustrate feature similarity analysis between various pre-trained backbones in accordance with an embodiment. FIGS. 12A through 12I show scatterplots of the centered kernel alignment (CKA) similarity index analysis when comparing the representation of the volumetric-CT dataset induced by different pre-trained backbones. Each panel corresponds to a different pair of pre-trained backbones (FIGS. 12A through 12C biomedical pairs; FIGS. 12D through 12F biomedical and ImageNet pairs; FIGS. 12G through 12I biomedical and random pairs). In each panel, each of the 768 dots represents the similarity score computed for the representations induced by the corresponding filter. A dot is red if it corresponds to one of the top 5% scores (and gray otherwise). The dashed lines show the average score measured for the color-corresponding set of dots.

In the second experiment, several embodiments assess the cross medical imaging modality of SLIViT. Four 2D biomedical-imaging datasets (Kermany, OrganMNIST, ChestMNIST, and Mixed (a dataset made up of images from all three biomedical datasets)) are used to pre-train four SLIViT models. Each model is initialized using ImageNet weights and then pre-trained on the respective 2D biomedical-imaging dataset. The Mixed-based SLIViT is pre-trained to classify one out of the total 29 classes included in these three 2D modalities (four for OCT, 11 for CT, and 14 for X-ray). Then for each volumetric-medical-imaging-learning task, each of the four pre-trained models is fine-tuned. As in the other pretraining experiments, using 2D OCT data in pre-training can provide an advantage in the 3D OCT classification tasks. Furthermore, in all other analyzed tasks, SLIViT can achieve better performance relative to the competitor benchmarks tested, regardless of the 2D biomedical-imaging dataset used for pre-training. This discovery further illustrates SLIViT's cross-modality and cross-dimensionality potencies in 3D biomedical-imaging learning tasks.

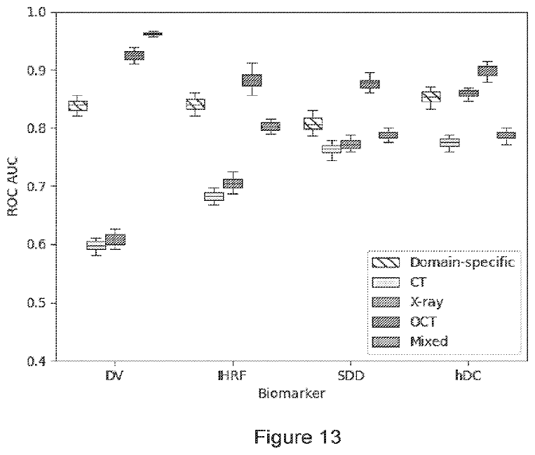

FIG. 13 illustrates 2D biomedical-imaging pre-training performance contribution for 3D OCT-related downstream learning tasks in accordance with an embodiment. FIG. 13 shows the ROC AUC scores on four volumetric-OCT single-task classification problems. Four SLIViT models are evaluated in every classification problem. Each SLIViT model is initialized with ImageNet weights and then pre-trained on a 2D biomedical-imaging dataset of a different modality. The considered modalities are CT, X-ray, OCT, and Mixed (containing all the images from the CT, X-ray, and OCT datasets). SLIVER-net's performance (Domain-specific) is borrowed from FIG. 3. The expected performance of a random model is 0.5. Box plot whiskers represent a 90% CI.

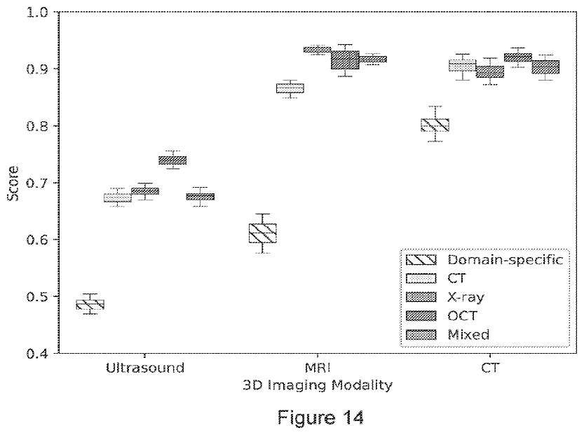

FIG. 14 illustrates 2D biomedical-imaging pre-training performance contribution for 3D non-OCT-related downstream learning tasks in accordance with an embodiment. FIG. 14 shows the performance R² scores for the volumetric ultrasound and MRI regression tasks (R²) and the volumetric CT classification task (ROC AUC). Four SLIViT models are evaluated in every learning problem. Each SLIViT model is initialized with ImageNet weights and then pre-trained on a 2D biomedical-imaging dataset of a different modality. The considered modalities are CT, X-ray, OCT, and Mixed (containing all the images from the CT, X-ray, and OCT datasets). The performance scores of the domain-specific methods are borrowed from FIG. 2. The expected R² and ROC AUC of a random model are zero and 0.5, respectively. Box plot whiskers represent a 90% CI.

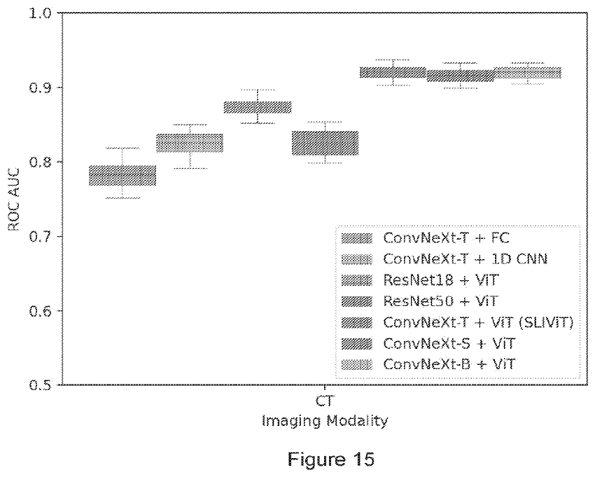

FIG. 15 illustrates architecture ablation study in accordance with an embodiment. FIG. 15 shows SLIViT's ROC AUC scores on a volumetric-CT classification task with different combinations for the feature-map extractor and feature-map integrator. The configuration used by the published version of SLIViT is ConvNeXt-T+ViT. The expected performance of a random model is 0.5. Box plot whiskers represent a 90% CI.

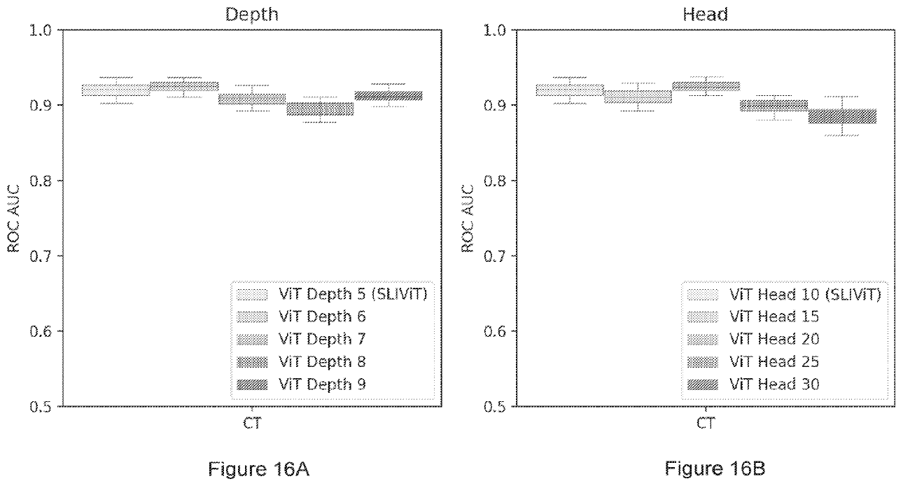

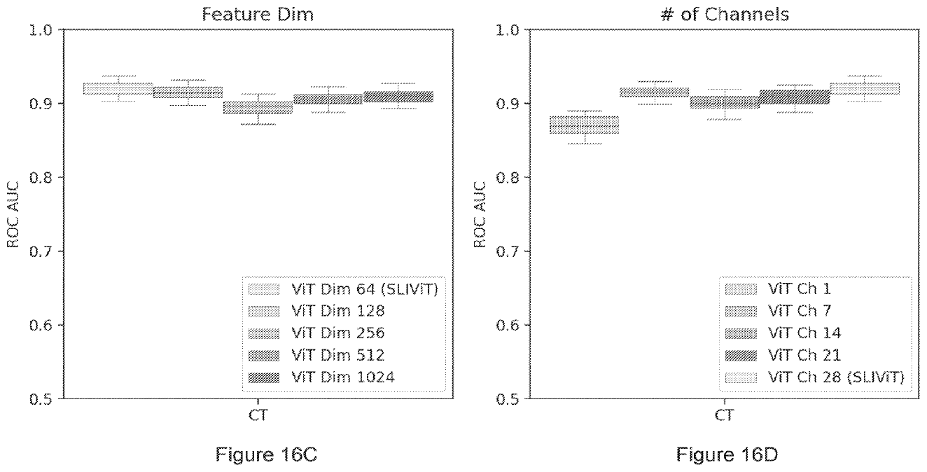

FIGS. 16A through 16D illustrate ViT hyperparameter tuning experiment in accordance with an embodiment. The figures show SLIViT's performance scores on a volumetric-CT classification task with different technical configurations for the ViT feature-map integrator. The configuration used by the published version of SLIViT is marked in each panel. The expected performance of a random model is 0.5. Box plot whiskers represent a 90% CI. FIG. 16A shows the depth. FIG. 16B shows the head. FIG. 16C shows the feature dim. FIG. 16D shows the number of channels.

### SLIViT Performances

Procuring tens of thousands of annotated 3D biomedical-imaging samples to train standard 3D vision models is expert-time prohibitive, impeding the full optimization of such models. The SLIViT processes in accordance with many embodiments can allow an accurate analysis of potentially any 3D biomedical-imaging dataset. SLIViT leverages a combination of deep vision modules and ‘prior knowledge’ from the 2D domain. This, in turn, allows it to be adept at 3D biomedical imaging learning tasks, in which the number of annotated training samples can be very limited. SLIViT outperforms domain-specific models.

SLIViT's effectiveness and generalizability can be demonstrated over several classification and regression problems in diverse biomedical domains (retinal, cardiac, and hepatic) across different 3D biomedical-imaging data modalities (OCT, echocardiograms, and MRI) against domain-specific and generic (fully-supervised- and self-supervised-based) methods. SLViT can be trained on less than 700 volumes in four independent binary classification learning tasks of retinal-disease risk factors with two independent 3D OCT datasets. SLIViT can be applied to heart function analysis tasks with echocardiogram datasets. SLIViT can be used for MRI datasets of 3D liver scans labeled with a corresponding hepatic fat content measurement. SLIViT can be used for analyzing pulmonary nodule malignancy screening using a CT dataset of thoracic scans. SLIViT can obtain on par performance to clinical specialists' assessment, and almost four orders of magnitude faster compared to the annotation procurement net time required by the specialists. SLIViT's learning ability robustness can be proved with randomly permuted volumes. Some embodiments use shuffled volumes dataset, which has little to no effect on SLIViT's performance, meaning that SLIViT is potentially agnostic to imaging protocol.

To facilitate reproducibility, generalizability, and applicability of SLIViT to various datasets, several embodiments avoided complex hyperparameter tuning and the usage of specialized hardware for training as required by other methods. The sizes of the different architectures can be set to available (standard) computational resources, and other hyperparameters are set to default values.

The utility of self-supervised pre-training has been validated in medical imaging learning tasks, however, its general translatability across domains remains unclear. According to several embodiments where a large-enough 2D labeled dataset is accessible and limited labeled volumes are available, the supervised pre-training approach is superior. This finding is supported by experiments for fine-tuning both in the same domain and across domains. SLIViT's pre-training strategy is flexible and can harness the utility of self-supervised approaches, such as REMEDIS. If one has access to an(other) unlabeled dataset of relevant medical images (whether 2D or 3D), then self-supervised pre-training SLIViT (either) as an alternative to (or followed/preceded by) supervised 2D OCT B-scans pre-training may further improve the model's performance. The end-to-end fine-tuning approach SLIViT takes is shown to attain better performance for self-supervised-based medical-imaging learning tasks. SLIViT already employs an optimized fine-tuning approach for a potential self-supervised-based avenue.

SLIViT is tested on 3D OCT scans, echocardiograms, 3D CT scans, and MRI volumes and can potentially be leveraged to analyze other types of volumetric medical imaging data modalities, such as 3D X-ray imaging. Such imaging data are inherently structured in the sense that they involve a limited assortment of objects and movements (typically shrinkage, dilation, and shivering). SLIViT is tailored to be adept at analyzing a series of biomedical frames created in a structured biomedical-imaging process and does not pretend to be proficient at learning problems of natural videos, such as action recognition tasks. Natural videos are inherently more complex, as the background may change, objects may flip, change color (due to shade), and even disappear (due to obfuscation). In addition, there is a plethora of natural video datasets that allow standard 3D-based vision models to be adequately tuned for natural video learning tasks. SLIViT could potentially be tweaked to perform well on natural videos as well, e.g., using a different feature-map extractor.

Several embodiments provide that multiple additional steps may be needed to deploy SLIViT in a clinical setting. Notably, the point of operation (tradeoff between precision and recall) is application specific and further optimization may be required to obtain optimal results at that point of operation. Point of operation may vary across clinicians. Moreover, additional evaluations of the models may be needed to ensure no systematic biases exist that would lead to increasing health disparities.

SLIViT provides an important step toward fully automating volumetric-biomedical-imaging annotation. The major leap happens under ‘real life’ settings of a low-number training dataset. SLIViT thrives by giving just hundreds of training samples for some tasks giving it an advantage over other 3D-based methods, in cases that are related to 3D biomedical-imaging annotation. Once a previously unknown disease-related risk factor is found and characterized, it could take months in order to train a specialist to be able to accurately annotate this recently discovered risk factor in biomedical images at scale. However, using a relatively small training dataset (that can be annotated within only a few working days of a single trained clinician), SLIViT could expedite the annotation process of other non-annotated volumes with an on par performance level of a clinical specialist.

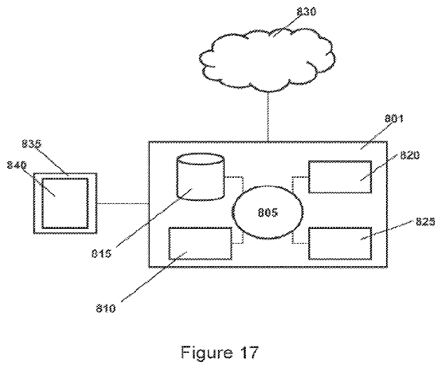

In some embodiments, the prediction processes can be performed with a computer system. FIG. 17 shows a computer system 801 that can be configured to implement any computing system disclosed in the present disclosure. The computer system 801 can comprise a mobile phone, a tablet, a wearable device, a laptop computer, a desktop computer, a central server, etc.

The computer system 801 includes a central processing unit (CPU, also “processor” and “computer processor” herein) 805, which can be a single core or multi core processor, or a plurality of processors for parallel processing. The CPU can be the processor as described above. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. In some cases, the communication interface may allow the computer to be in communication with another device such as the imaging device or audio device. The computer may be able to receive input data from the coupled devices for analysis. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

Methods and systems of the present disclosure can be implemented byway of one or more algorithms. An algorithm can be implemented by way of software upon execution by the central processing unit 805. The algorithm can, for example, partition a computer model of a part according to a hierarchy, receive user input data for modifying one or more parameters and produce a machine code.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The memory 810 can be a volatile or non-volatile, magnetic, semiconductor, tape, optical, removable, non-removable, or other type of storage device or tangible and/or non-transitory computer-readable medium that stores programs, such as the interactive slicer and operating system. Common forms of non-transitory media include, for example, a flash drive, a flexible disk, a hard disk, a solid state drive, magnetic tape or other magnetic data storage medium, a CD-ROM or other optical data storage medium, any physical medium with patterns of holes, a RAM, a PROM, and EPROM, a FLASH-EPROM or other flash memory, NVRAM, a cache, a register or other memory chip or cartridge, and networked versions of the same.

The memory 810 may store instructions that enable processor to execute one or more applications, such as the interactive slicer and operating system, and any other type of application or software available or executable on computer systems. Alternatively, or additionally, the instructions, application programs, etc. can be stored in an internal and/or external database (e.g., a cloud storage system—not shown) that is in direct communication with computing device, such as one or more databases or memories accessible via one or more networks (not shown). The memory 810 can include one or more memory devices that store data and instructions usable to perform one or more features provided herein. The memory 810 can also include any combination of one or more databases controlled by memory controller devices (e.g., servers, etc.) or software, such as document management systems, Microsoft SQL databases, SharePoint databases, Oracle™ databases, Sybase™ databases, or other relational databases. Data used in the slicing process such as hierarchies, rules for portioning a model corresponding to each hierarchy, valid range for some or all of the parameters, printer configurations, printer specifications, and the like may be stored in the one or more databases.

The computer system 801 may be communicatively connected to one or more remote memory devices (e.g., remote databases—not shown) through a network. The remote memory devices can be configured to store information that computer system 801 can access and/or manage. By way of example, the remote memory devices may be document management systems, Microsoft SQL database, SharePoint databases, Oracle™ databases, Sybase™ databases, Cassandra, HBase, or other relational or non-relational databases or regular files. Systems and methods provided herein, however, are not limited to separate databases or even to the use of a database.

The computer system 801 can communicate with one or more remote computer systems through the network 830. For instance, the computer system 801 can communicate with a remote computer system of a user. Examples of remote computer systems include personal computers, slate or tablet PC's, smart phones, personal digital assistants, and so on. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine-readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as the main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface 840 for providing, for example, a scanning interface or a footwear purchase interface. Examples of UI's include, without limitation, a graphical user interface (GUI) and web-based user interface.

Methods and systems of the present disclosure can be implemented byway of one or more algorithms. An algorithm can be implemented by way of software upon execution by the central processing unit 805.

### EXEMPLARY EMBODIMENTS

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### Example 1: SLIViT's Development and Analysis

SLIViT can be implemented in Python 3.8 using PyTorch v1.10.2, fast.ai v2.6.3, and scikit-learn v1.0.2 libraries.

### Example 1.1: Model Specifications

In many embodiments of the invention, the SLIViT framework contains a preprocessing step, a 2D ConvNeXt that serves as a feature-map extractor, and a vision transformer (ViT) that serves as a feature-map integrator. A ConvNeXt architecture has several complexities. The backbone of the tiny variant (ConvNeXt-T) with 256×256 image size can be used as SLIViT's feature-map extractor. An ablation study is done using the 3D CT dataset to evaluate different combinations for the feature-map extractor and feature-map integrator. The ViT-based feature-map integrator has few adjustments with respect to the original architecture, including using GeLu as the activation functions and initializing the positional embeddings as the number of the original slice. Several embodiments intentionally avoid complex hyperparameter tuning and usage of specialized hardware. The ViT's depth (#layers=5) are set according to the available computational resources to facilitate reproducibility, generalizability, and the applicability to other datasets. The ViT's width is governed by the number of 2D frames of the input volume.

Let N be the number of H×W 2D frames of an input image. Given an input W×H×N image, its N frames are resized (according to the ConvNeXt-T variant) and tiled into an image of size N*256×256. The manipulated image is then fed into the feature-map extractor which generates, in turn, an (N*8)×8×768 feature map. This 3D feature map is partitioned into N different 8×8×768 3D “patches” (corresponding to the terminology used in the original ViT paper41). Note that due to the convolution's locality property, each of these patches roughly corresponds to features obtained from a different frame. Each of the N patches is then flattened into a 1D vector (of length 8*8*768) and then tokenized into a vector of size 768 using a fully connected (FC) layer. The patch number (that essentially corresponds to an original slice number), is then added to each of the tokenized patches, and the results are then fed into the ViT (along with a class token of the same size). The ViT outputs N encoded values and a class token. The class token is then fed into another FC layer to generate final output. Using the 2D ViT as a feature-map integrator corresponds with the Factorized Encoder with ‘late fusion of depth information’ of the previously devised 3D ViT named ViViT, but is less complex than the 3D ViT.

### Example 1.2: Pre-Training

An ImageNet-1K pre-trained SLIViT-like feature-map extractor architecture, i.e., a ConvNeXt-T backbone, is used and appended to a subsequent FC layer to fit a four-category classification task. This SLIViT-backbone-like module is trained on the publicly available labeled Kermany Dataset. Training the feature-map extractor on the Kermany Dataset takes less than about 12 hours using a single NVIDIA Tesla V100 Volta GPU Accelerator 32 GB Graphics Card. Several sets of pre-trained weights are examined in this study. The pre-trained backbone weights are obtained from combining ImageNet initialization with additional pre-training on the Kermany Dataset (henceforth “combined weights”).

### Example 1.3: Per-Task Fine-Tuninq

Each of the SLIViT models used in the different experiments is initialized with the combined weights. The fine-tuning is done in an end-to-end fashion. Namely, rather than merely training the downstream feature-map integrator, while keeping the feature-map extractor frozen, the model's parameters are set as trainable, and are then fine-tuned (according to the dataset and task in question). The complex hyperparameter tuning is intentionally avoided to facilitate reproducibility and generalizability. Frames are resized into 256×256 pixels to fit SLIViT's backbone architecture and then, standard preprocessing transformations are applied (including contrast stretching, random horizontal flipping, and random resize cropping) using PyTorch's default values. Binary cross entropy and L1 norm are used as loss functions for the classification and regression tasks, respectively. In each experiment, excluding the ultrasound (in which the split is given), a random validation set is used for determining the convergence of the training process with the same loss function metric used for the test set evaluation. The model is optimized using the default fast.ai optimizer with the default parameters. The starting learning rate in each training procedure is chosen by fast.ai's learning rate finder and the model is fitted using the fit-one-cycle approach for faster convergence. The models are trained with four samples per batch and early stopping is set to five epochs, meaning that the training process continues until no improvement is observed in the validation loss for five consecutive passes on the whole training set. The model weights that achieve the lowest loss on the validation set during training are used for the test set evaluation. Weights & Biases is used for experiment tracking and visualizations of the training procedures.

### Example 1.4: Statistical Analysis

The performance of each trained model is evaluated (on the corresponding test set) using an appropriate metric score. The binary classification tasks are evaluated using area under the ROC and PR curves. The regression tasks are evaluated using the R2 metric. The test set predictions are calculated, and a 90% confidence interval (CI) is computed for each evaluated score using a standard bootstrapping procedure with 1,000 iterations. Briefly, let n denote the test set size, for each bootstrap iteration n samples are randomly drawn (with repetition) and based on the predictions of the sampled set a single score is obtained. Out of the 1,000 sampled-sets score distribution, the 50th and 950th ranked scores are selected to obtain the 90% CI. To compute the significance value of the difference between two given distributions (induced by two different models) a paired t-test on the distribution of differences between the sampled-set corresponding scores is computed (HA: 0). SLIViT's performance improvement can be significant if the paired t-test produced a p-value lower than 1e-3 subject to Bonferroni correction for multiple hypothesis testing.

### Example 1.5: Feature Similarity Analysis

To demonstrate the visual similarity across biomedical domains, the centered kernel alignment (CKA) similarity index is used. CKA allows measuring the similarity between the features extracted using any two neural-network layers for a given sample set. The output of the feature-map extractor (when initialized with different sets of weights) is compared, as it functions as the input for the feature-map integrator. Five of the backbone versions are used, each initialized with a different set of pre-trained weights. The sets include three 2D-medical-imaging-based weights (that are obtained by pre-training on the Kermany Organ{A,C,S}MNIST, and ChestMNIST datasets), ImageNet weights, and random weights. The CKA similarity scores are computed by projecting the volumes from the NoduleMNIST3D Dataset onto the feature space of each of the five models. For each of the projection pairs (excluding ImageNet-random pair), the CKA scores are computed between corresponding-slices projections of a given volume and averaged the results across slices and volumes. Two CKA distributions are considered for the comparison: the top-5%-scores distribution and the overall-scores distribution. The difference significance between the CKA of different pairs of models was assessed using a standard t-test (H_{A}: μ≠0).

### Example 1.6: Benchmark Specifications

Several baselines are used to benchmark SLIViT across the different data modalities. The baselines include SLIVER-net, two different types of 3D ResNet-R3D (unless stated otherwise) and R(2+1)D, 3D ViT, UniMiSS, EchoNet, 2D-Slice-CNN-based architecture. As SLIViT, models trained with the fit-one-cycle learning-rate scheduler. SLIVER-net is subjected to the same pre-training approach as SLIViT, namely, an ImageNet weights initialization followed by supervised pre-training on the Kermany Dataset. EchoNet performance is reproduced (on the same dataset). The (ImageNet-initialized) 2D-Slice-CNN-based are already optimized for MRI-based learning tasks and thus used as is. As for UniMiSS, the pre-trained MiT-22 variant that is shown to be best performative across all the tasks examined is used. Although other studies use benchmarks as is while optimizing their method (e.g., 19,28), several hyperparameter tuning experiments for the more generic methods (i.e., 3D ResNet and 3D ViT) are conducted to confirm that their default configurations are reasonable. The experiments comply with the following best practices. Given a dataset, the original validation and test sets are set aside. The original training set is split into sub-train, sub-validation, and sub-test, using the same proportions in the original split. The different examined hyperparameter configurations are evaluated using this split. For each configuration, the weights of the model with the lowest sub-validation loss are used to evaluate the performance on the sub-test set. Due to the heavy computational cost and limited resources, only a restricted number of hyperparameter configurations are examined, and only the classification task in the 3D CT dataset is considered. The hyperparameters examined for 3D ResNet are # of layers (18 and 50) and pre-training strategy (random weights and Kinetics40090 weights). The hyperparameters examined for 3D ViT are width (96 and 192) and depth (4 and 6). Neither configuration evaluations end up with significant improvements (on the sub-test set) and given the many heavy computations required for this study, the simplest (previously optimized) configuration is used. That is, randomly initialized 18-layer 3D ResNet and factorized spatiotemporal encoder 3D ViT (with default hyperparameters) to be the best performative variant26. In addition, the performance of a self-supervised-based SLIViT using the REMEDIS approach is examined. To this end, SLIViT is examined with ImageNet weights and then pre-trained on an unlabeled version of the Kermany Dataset, using SimCLR91 (REMEDIS' default learning scheme) as the self-supervised strategy.

### Example 2: Datasets

### Example 2.1: The Houston Dataset

1,128 patients were diagnosed with intermediate AMD in their scanned eye by clinical examination (Beckman Classification) at the Retina Consultants of Texas Eye Clinics between October 2016 and October 2020. This study was reviewed and approved by the Ethics Committee of Retina Consultants Texas (Houston Methodist Hospital, Pro00020661:1 “Retrospective Prospective Analysis of Retinal Diseases”). As the data collection was retrospective, a waiver of informed consent was granted. In case both eyes of a given patient were eligible, one eye was randomly included in the dataset. The dataset included Heidelberg Spectralis (HRA+Optical Coherence Tomography OCT SPECTRALIS; Heidelberg Engineering, Inc, Heidelberg, Germany) 6×6 mm (fovea centered, 10×10 degrees; 49 B-scans spaced 122 microns apart, ART=6) OCT volumes. The data were transferred to the Doheny Image Reading Research Laboratory (DIRRL) for imaging analysis and annotation of the structural OCT biomarkers for AMD progression. The AMD-biomarker analysis was conducted at the Doheny Image Reading Research Laboratory (DIRRL) in compliance with the Declaration of Helsinki and approved by the UCLA Institutional Review Board (IRB, Ocular Imaging Study, Doheny Eye Center UCLA). Cases with evidence of late stage of AMD and/or additional macular diseases or poor-quality imaging were excluded from the analysis. In total, 691 eyes (of 691 patients) were eligible for the biomarkers analysis. The annotations were procured by a senior clinical retina specialist. The recorded case frequency in the whole dataset was as follows: (1) 48.23% of the scans had drusen volume >0.03 mm3 within the 3 central mm2 (denoted DV); (2) 36.17% of the scans had intraretinal hyperreflective foci (denoted IHRF); (3) 31.45% of the scans had subretinal drusenoid deposits (SDD); and (4) 11.27% of the scans had hyporeflective drusen core (hDC). The positive-label frequencies of the test set were 47%, 43.5%, 52.8%, and 31.3%, respectively.

### Example 2.2: The SLIVER-Net Dataset

The SLIVER-net Dataset, which was originally used by Rakocz and others to tune and validate SLIVER-net, was collected from three independent medical centers between February 2013 and July 2016. The dataset included 1,007 OCT volumes each consisting of 97 B-scans (97,679 B-scans overall) collected from 649 subjects of the Amish general population, who had a record of at least one individual with AMD in the family history. Imaging was conducted at three clinical centers in Pennsylvania, Indiana, and Ohio under the supervision of investigators at the University of Pennsylvania (UPEN), University of Miami (MU), and Case Western Reserve University (CWRU), respectively. The research was approved by the institutional review boards (IRBs) of the respective institutions and all subjects signed written informed consent. All OCT B-scan volumes in this dataset were acquired with the Heidelberg Spectralis OCT using a scan pattern centered on the fovea (20°×20°; 97 B-scans; 512 A-scans per B-scans; ART 9). In order to fit the Houston Dataset trained model, each of the SLIVER-net Dataset volumes was down-sampled by taking every other B-scan, thus squeezing each volume to 49 B-scans. Also, to avoid aliasing, we applied an anti-aliasing filter on OCT volumes.

The positive-label frequencies in this dataset were 3.37%, 7.87%, 2.0%, and 2.67%, for DV, IHRF, SDD, and hDC, respectively. Although the annotations for this dataset included the eyes laterality, the scans themselves lacked the laterality obscuring the link between a scan to its annotation in case both eyes were scanned for a patient. To address this gap, the middle slice per volume was used to determine the laterality and trained a standard CNN on the Houston Dataset (that had the eyes laterality recorded). Using the trained network (97% accuracy on an external test set; not shown), the laterality for the SLIVER-net dataset scans was inferred when needed, that is, when both eyes of the same patient were scanned.

### Example 2.3: The Pasadena Dataset

The Pasadena Dataset established contained 205 3D OCT B-scan volumes (fovea centered, 10×10 degree, ART=5) collected from 205 individuals at the Doheny-UCLA Eye Centers in Pasadena between 2013 and 2022. This study was reviewed and approved by the IRB of the University of California, Los Angeles (UCLA IRB #15-000083). Informed consent was waived for study participants given the retrospective nature of the study. Each of the OCT volumes was acquired on the Heidelberg Spectralis HRA+Optical Coherence Tomography (OCT SPECTRALIS; Heidelberg Engineering, Inc, Heidelberg, Germany). Out of the 205 OCT volumes, 198 contained 97 B-scans and seven contained 49 B-scans. The OCT B-scans were independently annotated by ten DIRRL-certified clinical retina specialists: three seniors (expert retina specialists) and seven juniors. The ground truth for this dataset was determined by the senior retina specialists. Although the senior graders agreed in most cases, in the atypical case of disagreement, the ground truth was obtained by a majority vote of the senior graders' quorum. The positive-label frequencies in this dataset were 32.8%, 51.6%, 42.9%, and 12.5%, for DV, IHRF, SDD, and hDC, respectively.

### Example 2.4: The EchoNet-Dynamic Dataset

The EchoNet-Dynamic Dataset contains 10,030 echocardiograms (heartbeat ultrasound videos) obtained from 10,030 different individuals who underwent echocardiography between 2006 and 2018. Each echocardiogram was labeled with a continuous number (between zero and one) representing the ejection fraction (EF). The EF was obtained by a registered sonographer and further verified by a level 3 echocardiographer. The minimal EF in the dataset was 0.069 while the maximal was 0.97. The average EF was 0.558 with a standard deviation of 0.124. The dataset already set a random split for train, validation, and test sets of sizes 7,465 (74.43%), 1,288 (12.84%), and 1,277 (12.73%), respectively. In contrast to the other datasets used in this study, the number of frames (2D images) per video in the dataset was not constant but rather varied from 28 to 1,002 (with nearly 177 frames on average and a standard deviation of 58 frames). To standardize the data, 32 equally spaced frames per volume were sampled.

### Example 2.5: The United Kingdom Biobank Dataset

The United Kingdom Biobank (UKBB) Dataset of MRI imaging with Proton Density Fat Fraction (PDFF) measurements was downloaded on Jun. 7, 2022, from the UKBB repository. The UKBB is a widely studied population-scale repository of phenotypic and genetic information for roughly half a million individuals. At the time of the study, the UKBB made available 16,876 PDFF measurements acquired from a subset of the 54,606 total hepatic-imaging MRIs. The MRI data of each individual consisted of an unordered series of 36 imaging scans in DICOM format at 284 by 288 resolution (in-plane pixel spacing 9.3 mm) acquired from a single breath-hold session. Of the data available, a subset of 9,954 was identified White British individuals who were unrelated and possessed both the hepatic MRI and PDFF measurement. The individuals were further divided into train, validation, and test sets of sizes 5972 (60%), 1991 (20%), and 1991 (20%), respectively.

### Example 2.6: The NoduleMNIST3D Dataset

The NoduleMNIST3D Dataset61 is based on the Lung Image Database Consortium and Image Database Resource Initiative (LIDC/IDRI) Dataset of volumetric-CT imaging90. The dataset contains 1,633 scans each is (binary) labeled for nodule existence, with a positive-label frequency of 24.56%. The dataset was downloaded on Dec. 8, 202361. The dataset has a pre-defined random split for train, validation, and test sets of sizes 1,158 (70.91%) 165 (10.1%), and 310 (18.98%), with positive-label frequencies of 25.47%, 25.45%, and 20.65%, respectively.

### EXAMPLES

Example 1: A method to predict biomarkers in biomedical imaging, comprising: reshaping a plurality of three-dimensional images into a plurality of two-dimensional images by stacking a plurality of slices of said three-dimensional images on top of one another using a computer system; applying a pre-trained feature extractor to the plurality of two-dimensional images, wherein the pre-trained feature extractor independently operates on each of the plurality of two-dimensional images, and generates a plurality of feature maps; applying a convolutional neural network to operate across the plurality of feature maps, wherein the convolutional neural network produces a feature vector; and generating an output of biomarker prediction, wherein the prediction is a transformation of the feature vector; wherein the plurality of three-dimensional images is selected from a group consisting of: optical coherent tomography images, ultrasound images, magnetic resonance imaging images, and computed tomography images.

Example 2: The method of example 1, wherein the biomarker for optical coherent tomography images is selected from the group consisting of drusen volume (DV), intraretinal hyperreflective foci (IHRF), subretinal drusen deposits (SDD), hyporeflective drusen core (hDC), and any combinations thereof.

Example 3: The method of example 1 or 2, wherein the biomarker for ultrasound images comprises ejection fraction, cardiomyopathy, and a combination thereof.

Example 4: The method of example 1, or 2, or 3, wherein the biomarker for magnetic resonance imaging images comprises hepatic proton density fat fraction level.

Example 5: The method of any one of examples 1 to 4, wherein the biomarker for computed tomography images comprises nodule malignancy in thoracic cancer.

Example 6: The method of any one of examples 1 to 5, further comprising: obtaining a training dataset of images using the computer system; generating a first set of features for each image in the training dataset based upon object classification using the computer system; and training the feature extractor to learn relationships between the set of images in the training dataset and the first set of features in the training dataset using the computer system.

Example 7: The method of any one of examples 1 to 6, wherein the training dataset comprises a plurality of ImageNet dataset.

Example 8: The method of any one of examples 1 to 7, further comprising: obtaining an annotated training dataset comprising optical coherent tomography images using the computer system, wherein each of the optical coherent tomography images is annotated with at least one retinal disease risk factor; generating a second set of features for each annotated image in the annotated training dataset using the computer system; and training the feature extractor to learn relationships between each annotated image and the second set of features in the annotated training dataset using the computer system.

Example 9: The method of any one of examples 1 to 8, wherein the annotated training dataset comprises two-dimensional images of optical coherent tomography images.

Example 10: The method of any one of examples 1 to 9, wherein the two-dimensional images are fovea scans.

Example 11: The method of any one of examples 1 to 10, wherein the pre-trained feature extractor trained with the annotated training dataset of optical coherent tomography images is used for analyzing three-dimensional images selected from a group consisting of: optical coherent tomography images, ultrasound images, magnetic resonance imaging images, and computed tomography images, and generating biomarker predictions thereof.

Example 12: The method of any one of examples 1 to 11, wherein the plurality of slices is stacked linearly to form the two-dimensional image.

Example 13: The method of any one of examples 1 to 12, wherein the convolutional neural network comprises a vision transformer module.

Example 14: The method of any one of examples 1 to 13, wherein the feature vector transformation is a decision layer comprising at least two fully connected layers.

Example 15: A method of training a feature extractor, comprising: obtaining a training dataset of images using a computer system; generating a first set of features for each image in the training dataset based upon object classification using the computer system; and training a feature extractor model to learn relationships between the set of images in the training dataset and the first set of features in the training dataset using the computer system.

Example 16: The method of example 15, wherein the training dataset comprises a plurality of ImageNet dataset.

Example 17: The method of f example 15 to 16, further comprising: obtaining an annotated training dataset of optical coherent tomography images using the computer system, wherein each of the optical coherent tomography images is annotated with at least one retinal disease risk factor; generating a second set of features for each annotated image in the annotated training dataset using the computer system; and training the feature extractor model to learn relationships between each annotated image and the second set of features in the annotated training dataset using the computer system.

Example 18: The method of example 15, or 16, or 17, wherein the annotated training dataset comprises two-dimensional images of optical coherent tomography images.

Example 19: The method of any one of examples 15 to 18, wherein the two-dimensional images are fovea scans.

Example 20: The method of any one of examples 15 to 19, wherein the feature extractor trained with the annotated training dataset of optical coherent tomography images is used for analyzing three-dimensional images selected from a group consisting of: optical coherent tomography images, ultrasound images, magnetic resonance imaging images, and computed tomography images, and generating biomarker predictions thereof.

Example 21: The method of any one of examples 15 to 20, wherein the biomarker for optical coherent tomography images is selected from the group consisting of drusen volume (DV), intraretinal hyperreflective foci (IHRF), subretinal drusen deposits (SDD), hyporeflective drusen core (hDC), and any combinations thereof; wherein the biomarker for ultrasound images comprises ejection fraction, cardiomyopathy, and a combination thereof; wherein the biomarker for magnetic resonance imaging images comprises hepatic proton density fat fraction level; wherein the biomarker for computed tomography images comprises nodule malignancy in thoracic cancer.

### DOCTRINE OF EQUIVALENTS

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the terms “approximately” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

What is claimed is:

1. A method to predict biomarkers in biomedical imaging, comprising:
reshaping a plurality of three-dimensional images into a plurality of two-dimensional images by stacking a plurality of slices of said three-dimensional images on top of one another using a computer system;
applying a pre-trained feature extractor to the plurality of two-dimensional images, wherein the pre-trained feature extractor independently operates on each of the plurality of two-dimensional images, and generates a plurality of feature maps;
applying a convolutional neural network to operate across the plurality of feature maps, wherein the convolutional neural network produces a feature vector; and
generating an output of biomarker prediction, wherein the prediction is a transformation of the feature vector;
wherein the plurality of three-dimensional images is selected from a group consisting of: optical coherent tomography images, ultrasound images, magnetic resonance imaging images, and computed tomography images.

2. The method of claim 1, wherein the biomarker for optical coherent tomography images is selected from the group consisting of drusen volume (DV), intraretinal hyperreflective foci (IHRF), subretinal drusen deposits (SDD), hyporeflective drusen core (hDC), and any combinations thereof.

3. The method of claim 1, wherein the biomarker for ultrasound images comprises ejection fraction, cardiomyopathy, and a combination thereof.

4. The method of claim 1, wherein the biomarker for magnetic resonance imaging images comprises hepatic proton density fat fraction level.

5. The method of claim 1, wherein the biomarker for computed tomography images comprises nodule malignancy in thoracic cancer.

6. The method of claim 1, further comprising:
obtaining a training dataset of images using the computer system;
generating a first set of features for each image in the training dataset based upon object classification using the computer system; and
training the feature extractor to learn relationships between the set of images in the training dataset and the first set of features in the training dataset using the computer system.

7. The method of claim 6, wherein the training dataset comprises a plurality of ImageNet dataset.

8. The method of claim 6, further comprising:
obtaining an annotated training dataset comprising optical coherent tomography images using the computer system, wherein each of the optical coherent tomography images is annotated with at least one retinal disease risk factor;
generating a second set of features for each annotated image in the annotated training dataset using the computer system; and
training the feature extractor to learn relationships between each annotated image and the second set of features in the annotated training dataset using the computer system.

9. The method of claim 8, wherein the annotated training dataset comprises two-dimensional images of optical coherent tomography images.

10. The method of claim 9, wherein the two-dimensional images are fovea scans.

11. The method of claim 8, wherein the pre-trained feature extractor trained with the annotated training dataset of optical coherent tomography images is used for analyzing three-dimensional images selected from a group consisting of: optical coherent tomography images, ultrasound images, magnetic resonance imaging images, and computed tomography images, and generating biomarker predictions thereof.

12. The method of claim 1, wherein the plurality of slices is stacked linearly to form the two-dimensional image.

13. The method of claim 1, wherein the convolutional neural network comprises a vision transformer module.

14. The method of claim 1, wherein the feature vector transformation is a decision layer comprising at least two fully connected layers.

15. A method of training a feature extractor, comprising:
obtaining a training dataset of images using a computer system;
generating a first set of features for each image in the training dataset based upon object classification using the computer system; and
training a feature extractor model to learn relationships between the set of images in the training dataset and the first set of features in the training dataset using the computer system.

16. The method of claim 15, wherein the training dataset comprises a plurality of ImageNet dataset.

17. The method of claim 15, further comprising:
obtaining an annotated training dataset of optical coherent tomography images using the computer system, wherein each of the optical coherent tomography images is annotated with at least one retinal disease risk factor;
generating a second set of features for each annotated image in the annotated training dataset using the computer system; and
training the feature extractor model to learn relationships between each annotated image and the second set of features in the annotated training dataset using the computer system.

18. The method of claim 17, wherein the annotated training dataset comprises two-dimensional images of optical coherent tomography images.

19. The method of claim 18, wherein the two-dimensional images are fovea scans.

20. The method of claim 17, wherein the feature extractor trained with the annotated training dataset of optical coherent tomography images is used for analyzing three-dimensional images selected from a group consisting of: optical coherent tomography images, ultrasound images, magnetic resonance imaging images, and computed tomography images, and generating biomarker predictions thereof.

21. The method of claim 20, wherein the biomarker for optical coherent tomography images is selected from the group consisting of drusen volume (DV), intraretinal hyperreflective foci (IHRF), subretinal drusen deposits (SDD), hyporeflective drusen core (hDC), and any combinations thereof; wherein the biomarker for ultrasound images comprises ejection fraction, cardiomyopathy, and a combination thereof; wherein the biomarker for magnetic resonance imaging images comprises hepatic proton density fat fraction level; wherein the biomarker for computed tomography images comprises nodule malignancy in thoracic cancer.
